(19) United States
(12) Patent Application Publication
(10) Pub. No.: US 2023/0203220 A1
(43) Pub. Date: Jun. 29, 2023

(54) COPOLYMERS FOR INTRACELLULAR THERAPEUTIC NUCLEIC ACID PAYLOAD DELIVERY

(71) Applicant: Regents of the University of Minnesota, Minneapolis, MN (US)

(72) Inventors: Theresa Reineke, Vadnais Heights, MN (US); Ngoc Le, Minneapolis, MN (US); Zhe Tan, Secaucus, NJ (US); Ramya Kumar, Saint Paul, MN (US)

(21) Appl. No.: 18/000,258

(22) PCT Filed: May 28, 2021
(86) PCT No.: PCT/US2021/070634

§ 371 Date: Nov. 29, 2022

Related U.S. Application Data
(60) Provisional application No. 63/031,996, filed on May 29, 2020.

(51) Int. Cl. C08F 220/20; C08F 220/28; C08F 220/34; C08F 230/02; A61K 9/51

(52) U.S. Cl. CPC C08F 220/20; C08F 220/286; C08F 220/34; C08F 230/02; A61K 9/5138; C08F 2438/03

(57) ABSTRACT

A compound includes a polymer associated with a biological agent. The polymer has a first (meth)acryl monomeric unit with a cationic functional group R1 and a second (meth)acryl monomeric unit with a neutral hydrophilic functional group R2. The cationic functional group R1 is chosen from amino groups and alkylamino groups, and the neutral functional group R2 is chosen from polyethylene glycol (PEG), hydroxyl (OH), phosphorylcholine (PC), and mixtures and combinations thereof.

### GOVERNMENT INTEREST

This invention was made with government support under Grant No. N660011824041 awarded the Department of Defense/Defense Advanced Research Projects Agency (DARPA). The government has certain rights in the invention.

### BACKGROUND

Genome editing based on clustered regularly interspersed palindromic repeats (CRISPR) technology has transformed the therapeutic landscape for diseases wherein the deletion, insertion or repair of genetic sequences can restore healthy cellular states. Recently, clinical trials of investigational gene therapeutics for p-thalassemia and sickle cell disease suggest that safe and efficacious treatment is possible using CRISPR-based genome editing technology. Additional clinical trials are underway to develop CRISPR-based therapeutics for debilitating conditions such as Duchenne's muscular dystrophy (DMD), Leber congenital amaurosis (LCA) and for chimeric antigen receptor T-cell (CAR-T) therapies for cancer.

Despite the vast curative potential of CRISPR, widespread clinical deployment faces an uncertain outlook due to excessive reliance on engineered viral vectors, which can be used to deliver therapeutic biomacromolecule payloads such as, for example, messenger RNA (mRNA), plasmid DNA (pDNA) and small interfering RNA (siRNA). However, the high costs, lengthy delays and regulatory challenges involved in manufacturing clinical grade viruses at scale for large patient populations have imposed severe logistical bottlenecks. In addition to manufacturing and regulatory delays, the cargo capacity of viral vectors is limited, and this size ceiling is particularly problematic in the context of bulky multi-component CRISPR cargoes.

Although advances in virus manufacturing have minimized occurrences of carcinogenic mutations, genomic integration and fatal systemic inflammatory responses, these risks are amplified when repeated dosing or large dosages are involved. For CRISPR therapeutics to become safe, scalable and affordable, there is a need to identify synthetic substitutes for viral carriers.

Polymeric gene delivery vehicles have been used in clinical gene therapy due to their versatility, relative low production cost, and low immunogenicity. Synthetic polymers have been used to deliver biomacromolecule payloads such as, for example, pDNA, RNP, and the like, due to their versatility, low toxicity, and the ability to encapsulate large payloads. Some recent examples indicate that synthetic polymer-based systems achieved biomacromolecule based gene delivery and gene editing both in vitro and in vivo.

For example, in aqueous physiological solutions, cationic polymers can spontaneously bind with negatively charged pDNA and form interpolyelectrolyte complexes. These complexes are predominately internalized by various endocytic routes, followed by cargo release from these vesicles inside the cells via different proposed mechanisms, and subsequent entry into the cell nucleus to promote gene expression. Compared to viral vehicles, polymeric delivery systems typically have lower delivery efficiency, and various optimization strategies can be used to improve this parameter such as, for example, changing the cationic moieties on polymers, adding targeting ligands, and installing responsive monomers, which can improve uptake efficiency and help to balance transfection efficiency and cytotoxicity.

In one example, polyplexes formed with block copolymers consisting of distinct hydrophilic and cationic blocks compact pDNA, and have been shown to promote colloidal stability in biological media in addition to high transfection efficiency. Polymers are well established delivery vehicles for small molecule drugs and have demonstrated efficient delivery of siRNA, and pDNA delivery for gene silencing and transient transfection, respectively. However, their utility in genome editing is relatively underexplored.

In another example, CRISPR (clustered, regularly interspaced, short palindromic repeats)/Cas9 (CRISPR-associated protein 9)-based genome editing has rapidly emerged as a multi-faceted technology to enable gene insertion, deletion, activation, suppression, and even single base editing of target genes within the nucleus of any cell. This highly efficient and facile technique has broad utility from white biotechnology and agriculture to biomedical research, pharmaceutics, and regenerative medicine.

Currently, the CRISPR/Cas9 system can be delivered in vitro, ex vivo, and in vivo in three different payload forms: i) pDNA that encodes Cas9 protein and/or sgRNA ii) mRNA that encodes for Cas9 nuclease and a separate sgRNA, or iii) a ribonucleoprotein (RNP) that consists of recombinant Cas9 protein precomplexed directly with a sgRNA. While engineered viruses have shown exceptional delivery efficiency and expression of Cas9 protein in cells, limitations such as immunogenicity and size restrictions in packaging exist. Physical delivery methods such as electroporation and microinjection are known to cause cell damage or death and are challenging to apply to a large population of cells/tissues.

CRISPR-Cas9 pDNA needs to enter the cellular nucleus to express, and consistent expression produces an overabundance of Cas9 protein, which can lead to increased off-target editing and mutagenesis. Researchers have utilized the CRISPR/Cas9 system in mRNA form to circumvent the barrier of nuclear entry, which has been reported with polymer-based nanoparticles. However, sgRNA often needs to be delivered separately, presenting challenges in trafficking kinetics of different payloads.

Direct delivery of CRISPR/Cas9 ribonucleoprotein (RNP), on the other hand, has several benefits, including precision in endonuclease dosing and potential to avoid uncontrolled integration of the transgene into the cellular genome. While different CRISPR/Cas9 RNP delivery systems have been recently explored, such as lipid-based nanoparticles, gold nanoparticles, cell penetrating peptides, and other hybrid nanostructures, the mechanisms of payload encapsulation and the resultant complexes are generally not quantitatively understood/characterized. Polymers offer a well-documented pharmaceutically-relevant platform that have been underexplored for RNP encapsulation and delivery, and only limited number of reports have been presented, likely due to the inherent structural, charge, and binding differences of plasmid and protein-based payloads.

Designing novel and efficient polymer-based pDNA and RNP delivery vehicles, as well as improving the fundamental understanding of polymer-cargo complex composition and architecture on pDNA and protein loading and delivery efficiency, are necessary for advanced applications.

### SUMMARY

Developing biomaterials that deliver gene therapeutics is a complex design challenge spanning multiple length scales and time horizons. As a first step, delivery vehicles are expected to condense the CRISPR payloads (for example, mRNA, pDNA or ribonucleoproteins (RNP)), which can vary widely in their lengths, topologies, physical characteristics and biological mechanisms, into discrete nanosized polyelectrolyte complexes termed polyplexes. Upon administration, polyplexes must navigate both extracellular barriers such as serum DNAases (or RNAases) and reticuloendothelial system clearance, as well as intracellular barriers such as endosomal interrogation and lysosomal degradation. Finally, the cargo must be released within the spatiotemporal window that is optimal for payload translocation to the nucleus, where they can undergo further processing and realization of targeted edits. In addition to meeting high standards for safety, efficiency and cost-effectiveness, synthetic vectors must minimize immune activation and cellular toxicity. Engineering materials for gene delivery is a multivariate optimization process wherein even small deviations of material properties from the optimum may be poorly tolerated, leading to high rates of failure.

Because synthetic vector development demands precise control over interfacial properties, polymer chemistry is uniquely suited to optimize carriers of therapeutic biological cargoes. Cationic polymers are versatile alternatives to engineered viruses, but rapid clinical deployment hinges on efficient exploration of a vast chemical design space. The roles of polymer composition, length, architecture and other physicochemical attributes are typically probed through tedious “one-polymer-at-a-time” approaches, resulting in low rates of discovery.

Polymers open up large design spaces within which architecture, size, composition, basicity, surface charge and hydrophilicity can be systematically optimized to yield functional gene carriers. By incorporating a handful of distinct monomers in different proportions, architectures and lengths, almost infinite structurally unique design possibilities can be generated. Polymer chemists can produce portfolios of myriad macromolecules on demand, but the subsequent search for structures that elicit the desired biological responses is often more challenging than the synthetic effort itself. Since the biological milieu in which payloads are delivered by polymeric vectors is inherently complex, ab initio prediction of gene editing efficiency directly from polymer structure is difficult. With neither theoretical models nor heuristic knowledge providing reliable guidance, it is difficult to explore the polymer design space in a manner that minimizes experimental effort and accelerates material discovery.

In general, the present disclosure is directed to an interpolyelectrolyte complex including a copolymer associated with at least one biomacromolecular payload such as, for example, pDNA, RNP, and the like. The interpolyelectrolyte complexes (also referred to herein as polyplexes) are internalized by a cell via various endocytic routes, the biomacromolecular payload is released inside the cell, and the payload subsequently enters the cell nucleus to promote gene expression. The copolymers disclosed herein thus provide a polymeric scaffold that provides a well-defined host configured to bind with biological macromolecular agents and facilitate intracellular delivery thereof. The copolymers have physiochemical properties such as, for example, composition, molecular weight, ζ-potential, pKa, polyplex diameter, nucleic acid condensation, and combinations thereof selected for efficient nucleic acid payload delivery using, for example, a CRISPR/Cas9 delivery process. The copolymers and the polyplexes including the copolymers also have good gene editing efficiency, cellular internalization, cytotoxicity, and combinations thereof.

In one embodiment, the polyplexes include a copolymer with a backbone having a first (meth)acryl monomeric unit with a cationic functional group and a second (meth)acryl monomeric unit with a neutral hydrophilic functional group. In some embodiments, the copolymers include a first cationic monomeric unit of 2-(diisopropylamino) ethyl methacrylate (DIPAEMA) and a second neutral monomeric unit of hydroxy ethyl methacrylate (HEMA).

In another aspect, the present disclosure is directed to a method for identifying copolymers suitable for use in biomacromolecular payload delivery for gene editing procedures. In this method, combinatorial polymer design, parallelized experimental workflows, and statistical models were applied to discover high-performing polymeric vehicles suitable for efficient nucleic acid payment delivery using the CRISPR/Cas9 delivery process. A chemically diverse library of statistical copolymers identified using these models were then polymerized using a reversible addition fragmentation-transfer polymerization (RAFT) process with cationic monomers spanning a broad range of basicities, and co-monomers with varying degrees of hydrophilicity. High-throughput screening of gene editing outcomes was accompanied by extensive evaluation of selected physicochemical properties of the copolymers such as, for example, composition, molecular weight distribution, ζ-potential, pKa, polyplex diameter and payload binding affinity.

In another example of this method, polymeric vehicles were identified using combinatorial polymer design for co-delivery of pDNA repair templates along with RNP payloads as suitable for precise homology-directed repair (HDR)-mediated gene insertion. The polymeric vehicles have been shown capable of co-delivery of both RNPs and pDNA, despite the very distinct molecular attributes of these payloads. Several therapeutic applications, such as engineering T-cells to recognize tumor-associated antigens and initiate anti-tumoral responses, require challenging genomic modifications wherein extremely large DNA sequences (>1 kb) must be inserted via HDR. Gene repair through HDR retains strong therapeutic relevance since it is the best way to replace a certain gene variant with another in an error-free manner, the combinatorial method of the present disclosure identified copolymers that can package and deliver both payloads within a single polyplex. The polymeric vehicles can promote spatial and temporal co-localization of both sets of editing machinery, which can improve the efficiency of the HDR pathway for gene repair.

In some embodiments, the copolymers identified using the methods of the present disclosure outperformed state-of-the-art commercial transfection reagents, achieving nearly 60% editing efficiency via non-homologous end-joining.

The methods of the present disclosure can aid in discovering polymers that may otherwise be inaccessible to chemical intuition and yield model-derived design insights that will provide valuable synthetic guidance for future libraries.

In one aspect, the present disclosure is directed to a compound including a polymer associated with a biological agent. The compound includes a copolymer having a first (meth)acryl monomeric unit with a cationic functional group R₁ and a second (meth)acryl monomeric unit with a neutral hydrophilic functional group R₂; wherein the cationic functional group R₁ is chosen from amino groups and alkylamino groups, and the neutral functional group R₂ is chosen from polyethylene glycol (PEG), hydroxyl (OH), phosphorylcholine (PC), and mixtures and combinations thereof.

In another aspect, the present disclosure is directed to a method, including: selecting a cationic monomeric unit chosen from amino ethyl methacrylate (AEMA), 2-(diethylamino) ethyl methacrylate (DEAEMA), 2-dimethylamino ethyl methacrylate (DMAEMA), and 2-(diisopropylamino)ethyl methacrylate (DIPAEMA), and mixtures and combinations thereof; selecting a neutral monomeric unit chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof; reacting the cationic monomeric unit and the neutral monomeric unit to synthesize a copolymer; and associating the copolymer with a biomacromolecule to form a polyplex.

In another aspect, the present disclosure is directed to method that includes applying to a cell a composition including an aqueous pharmaceutically acceptable liquid carrier and a copolymer. The copolymer includes a cationic monomeric unit chosen from amino ethyl methacrylate (AEMA), 2-(diethylamino) ethyl methacrylate (DEAEMA), 2-dimethylamino ethyl methacrylate (DMAEMA), and 2-(diisopropylamino)ethyl methacrylate (DIPAEMA), and mixtures and combinations thereof; and a neutral monomeric unit chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof. A biological payload is associated with the copolymer, wherein the biological payload is chosen from pDNA, RNP, and mixtures and combinations thereof. The biological payload is delivered into the cell.

In another aspect, the present disclosure is directed to a method, including repairing DNA of a cell with a non-homologous end-joining (NHEJ) procedure. The NHEJ procedure includes administering to the cell a non-viral polyplex including a copolymer. The copolymer includes a cationic monomeric unit chosen from amino ethyl methacrylate (AEMA), 2-(diethylamino) ethyl methacrylate (DEAEMA), 2-dimethylamino ethyl methacrylate (DMAEMA), and 2-(diisopropylamino)ethyl methacrylate (DIPAEMA), and mixtures and combinations thereof; and a neutral monomeric unit chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof. A biological payload including RNP is associated with the copolymer. The RNP is delivered into the cell to repair the DNA of the cell.

In another aspect, the present disclosure is directed to a method including repairing DNA of a cell with a homology-directed repair (HDR) procedure. The HDR procedure includes administering to the cell a non-viral polyplex, which includes a copolymer, the copolymer including: a cationic monomeric unit chosen from amino ethyl methacrylate (AEMA), 2-(diethylamino) ethyl methacrylate (DEAEMA), 2-dimethylamino ethyl methacrylate (DMAEMA), and 2-(diisopropylamino)ethyl methacrylate (DIPAEMA), and mixtures and combinations thereof; and a neutral monomeric unit chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof. A biological payload is associated with the copolymer, wherein the biological payload is chosen from pDNA, RNP and mixtures and combinations thereof. The biological payload is delivered into the cell to repair the DNA of the cell.

In another aspect, the present disclosure is directed to a method including administering to a cell a non-viral transfection agent. The non-viral transfection agent includes a copolymer having a cationic monomeric unit chosen from amino ethyl methacrylate (AEMA), 2-(diethylamino) ethyl methacrylate (DEAEMA), 2-dimethylamino ethyl methacrylate (DMAEMA), and 2-(diisopropylamino)ethyl methacrylate (DIPAEMA), and mixtures and combinations thereof; and a neutral monomeric unit chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof. A pDNA payload is associated with the copolymer, and the pDNA is delivered into the cell to repair the DNA of the cell.

In another aspect, the present disclosure is directed to a non-viral polyplex including a copolymer and a biological agent associated with the copolymer. The copolymer includes a cationic monomeric unit chosen from amino ethyl methacrylate (AEMA), 2-(diethylamino) ethyl methacrylate (DEAEMA), 2-dimethylamino ethyl methacrylate (DMAEMA), and 2-(diisopropylamino)ethyl methacrylate (DIPAEMA), and mixtures and combinations thereof; and a neutral monomeric unit chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof. A biological agent chosen from pDNA, RNP, and mixtures and combinations thereof is associated with the copolymer.

In another aspect, the present disclosure is directed to a method for selecting a copolymer suitable for delivering a nucleic acid payload into a cell. The method includes: synthesizing a library of copolymers, wherein the copolymers in the library have a (meth)acrylate monomeric unit with a cationic functional group and a (meth)acrylate monomeric unit with a neutral hydrophilic functional group; complexing each of the copolymers in the library with a biomacromolecular payload to form a corresponding library of polyplexes, wherein the payloads in the polyplexes are chosen from plasmid DNA (pDNA), ribonucleoprotein (RNP), and mixtures and combinations thereof, screening each of the polyplexes in the library of polyplexes to determine a delivery efficiency into the cell, wherein the screening includes: determining at least one physiochemical property of the copolymers in the polyplexes, and determining at least one biological response of the polyplexes; correlating the at least one physiochemical property with the at least one biological response to generate a structure-function map; and selecting the copolymer from the structure-function map.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1F provide a schematic overview of an embodiment of a workflow used in the present disclosure.

FIG. 1A illustrates a synthesis of a combinational library of copolymers.

FIGS. 1B-1C illustrate that the copolymers in the library of FIG. 1A were assembled with either pDNA or RNP payloads to form polyplexes to identify preferred or “hit” copolymer candidates in the library.

FIG. 1D illustrates that delivery efficiency of the hit copolymers and polyplexes of FIGS. 1B-1C was screened using high-throughput biological assays.

FIG. 1E illustrates that in parallel with the screening of FIG. 1D, the copolymers and polyplexes of FIG. 1B-1C were physiochemically characterized, along with evaluation of internalization and toxicity.

FIG. 1F illustrates that statistical learning tools were deployed to mine experimental datasets and generate structure-function maps correlating copolymer and polyplex attributes and properties to key biological responses.

FIG. 2 is a summary of a combinatorially designed library of polymers synthesized using RAFT polymerization according to the present disclosure. Cationic monomers spanning a broad range of pKa values, were copolymerized with neutral monomers of varying hydrophilicity. The targeted cationic monomer incorporation was varied systematically from 0% to 100% in 25% increments.

FIG. 3A is a comparative plot of NMR analysis for the p(DIPAEMA-st-HEMA) series of copolymers in Table 1, which indicated that compositional control of the copolymerization reaction could be carried out merely by modulating monomer feed ratios.

FIG. 3B includes SEC chromatograms of the four cationic copolymers of the p(DIPAEMA-st-HEMA) series of copolymers in Table 1, which revealed that narrow molecular weight distributions were obtained across the entire compositional range (100% cationic to 25% cationic).

FIG. 3C is a plot of pKa values compared for the four cationic monomers in the p(DIPAEMA-st-HEMA) series of copolymers in Table 1. Changes in pH have been plotted as a function of the degree of deprotonation (0).

FIG. 3D is a plot of reactivity ratios of DIPAEMA and HEMA estimated using the Mayo-Lewis kinetic model. While DIPAEMA (rDIPAEMA=1) displayed equal preference for both cross-propagation and self-propagation, HEMA (rHEMA=2.7) had a slight preference for the latter.

FIG. 3E is a plot of electrophoretic mobilities of polymers were measured in PBS to monitor changes in ζ-potential with decreasing cationic monomer incorporation for the p(DIPAEMA-st-HEMA) series of copolymers in Table 1.

FIG. 4A is an overview of a screening process according to the present disclosure that can be used to select copolymers for use in transfection processes. In the HEK293 TLR cell line, if 100% editing efficiency were achieved via NHEJ, about ⅓rd of cells would produce mCherry, and mCherry expression levels was adopted as an indirect measure of editing efficiency. To quantify mCherry expression and gene editing outcomes with high throughput, while ensuring adequate sensitivity to low gene editing efficiencies, image cytometry was employed in tandem with high-content image analysis software pipelines.

FIG. 4B is a series of plots illustrating that RNP delivery was assessed at two formulation ratios (N/P of 1 and 2) across the entire library of copolymers and polyplexes. At the end of the screening study, p(DIPAEMA52-st-HEMA50), also referred to herein as DIP50HEMA50, emerged as the copolymer in the library with the best predicted performance.

FIG. 5A is a schematic illustration of plate-reader assays developed to screen the polymer library for candidates that could efficiently complex and deliver plasmid DNA (pDNA). Polyplexes were formulated at three N/P ratios during the screening studies and GFP expression resulting from transient transfection was compared.

FIG. 5B shows a correlation chart used to determine that while (DIPAEMA₅₂-st-HEMA₅₀) (DIP50H50) resulted in substantial GFP production within transfected cells at all three N/P ratios studied, p(DIPAEMA₆₁-st-HEMA₃₃) (DIP75H25) was effective only at the highest N/P ratio of 20.

FIG. 6A is a plot of mCherry expression in cells transfected with unpackaged ribonucleoprotein and polyplexes formed with the hit polymer, DIP50H50. The scale bar is 100 μm.

FIG. 6B shows representative flow cytometry traces, gated for single live cells, performed to benchmark the hit polymer against four commercial transfection reagents. At N/P ratios of 1, 1.5 and 2, the hit polymer resulted in higher mCherry expression than LPF CRISPRMAX and JetCRISPR.

FIG. 6C is a plot of NHEJ editing measured by Sanger sequencing and TIDE assay. Sanger sequencing validates observations from flow cytometry, establishing that the hit polymer outperformed all commercial controls by achieving 58% efficiency.

FIG. 6D shows representative chromatograms from cells treated with DIP50H50/RNP polyplexes at the highest N/P ratio. Chromatograms highlight the protospacer adjacent motif (red dotted line) and the sgRNA binding region (black solid line).

FIG. 6E is a plot of ζ-potential measurements of unpackaged RNPs and polyplexes formulated at various N/P ratios.

FIG. 6F is a representation of gel migration assays showing small amounts of unbound RNP in the polyplexes, suggesting that the binding between DIP50H50 and the RNP is moderate.

FIG. 6G is a plot of dynamic light scattering measurements (n=3 to 5) of RNP and polyplexes that yielded monomodal size distributions.

FIG. 7A is a schematic diagram of NHEJ and HDR editing pathways. While delivery of RNP alone promotes gene knock-out through NHEJ, co-delivery of plasmid DNA donor and RNP leads to gene knock-in via HDR.

FIG. 7B is a correlation chart used for optimization of formulation conditions for co-delivering RNP and donor DNA payloads. The total amount of nucleic acid was kept constant at either 1.5 μg or 2 μg per well, while the weight ratio of single guide RNA (sgRNA) and donor DNA was varied from 2:1 to 1:5. The optimal formulation was identified as 2 μg nucleic acid loading using a 1:2 w/w mixture of sgRNA and DNA, where HDR editing (quantified using GFP) was maximized.

FIG. 7C shows plots of fluorescence microscopy of HEK cells that underwent HDR-mediated genome editing while transfected as an unpackaged payload or using either Lipofectamine 2000 or the hit polymer as delivery vehicles. Scale bar is 100 μm. The HDR editing frequency effected by LPF 2000 was three-fold greater than the hit polymer.

FIG. 7D shows flow cytometry traces highlighting mCherry positive cell populations (representative of cells edited via the NHEJ pathway) and GFP positive cells (that underwent HDR).

FIG. 8A is a loading plot of that describes the contribution of each of the 9 descriptors to the top three PCs and whether the contribution is positive (light) or negative (dark).

FIG. 8B shows scatterplots of NHEJ editing results along the two main PCs.

FIG. 8C is a summary correlation chart of cellular viability and spCas9 uptake measured across the polymer library.

FIG. 8D shows random forest ensembles used to map physicochemical descriptors to three key biological responses: RNP transfection, cell viability and spcas9 internalization. While editing efficiency is highly dependent on hydrophobicity-associated parameters, toxicity is driven by polyplex diameter and protonation-dependent parameters such as ζ-potential, RNP binding and pKa.

FIG. 9A is a schematic representation of a flow cytometry process used to screen the polymer library for candidates that could efficiently complex and deliver plasmid DNA (pDNA). Polyplexes were formulated at three N/P ratios during the screening studies and GFP expression resulting from transient transfection was compared.

FIG. 9B includes plots for selected polyplexes illustrating that p(DIPAEMA52-st-HEMA50) or DIP50H50 resulted in the highest GFP production within transfected cells with the lowest toxicity among the library.

FIG. 10A(1) shows a microscopic examination of transiently transfected cells, which indicated that GFP expression was much higher in cells transfected using the hit polymer DIP50H50, compared to unpackaged plasmid DNA. The scale bar was 400 μm. From flow cytometric measurements, the pDNA delivery achieved by the hit polymer appeared to comparable to the transfection outcomes of two commercial transfection reagents, Lipofectamine 2000 and JetPEI. FIG. 10A(2) is a plot showing three replicates that were performed, and error bars represent two standard deviations.

FIG. 10B shows representative flow cytometry traces (gated for single live cells).

FIG. 10C is a plot of ζ-potential measurements of unpackaged pDNA and polyplexes formulated at various N/P ratios.

FIG. 10D shows strong binding between pDNA and DIP50HEMA50 was observed during gel migration assays, since no unbound pDNA was detected at all N/P ratios studied.

FIG. 10E shows dynamic light scattering measurements (n=3) of pDNA and polyplexes.

FIGS. 11A-11B are plots of DLS measurements of pDNA complexes formulated using DIPAEMA based copolymers.

FIG. 12 is a summary of gel electrophoresis assays where N/P ratios of 5, 10 and 20 were evaluated across the entire polymer library. Polyplex formulations where pDNA migration was observed are depicted in lighter shades while those that retained plasmids are shown in darker shades. Intermediate binding behavior where both retention and migration were observed, are shown as white.

FIG. 13A is a schematic of the NHEJ and HDR editing pathways utilized in Example 3. While delivery of RNP alone promotes gene knock-out through NHEJ (measured via mCherry), co-delivery of plasmid DNA donor and RNP leads to gene knock-in via HDR (measured via GFP).

FIG. 13B shows plots of optimization of formulation conditions for co-delivering RNP and donor DNA payloads. The total amount of nucleic acid was kept constant at either 1.5 or 2 μg per well while the weight ratio of single guide RNA (sgRNA) and donor DNA was varied from 2:1 to 1:5. The optimal formulation was identified as 2 μg nucleic acid loading using a 1:2 w/w mixture of sgRNA and DNA, where HDR editing (quantified using GFP) was maximized.

FIG. 13C shows fluorescent micrographs of HEK cells that underwent HDR-mediated genome editing while transfected as an unpackaged payload or using either Lipofectamine 2000 or the hit polymer as delivery vehicles. Scale bar is 100 μm. The HDR editing frequency effected by LPF 2000 was three-fold greater than the hit polymer.

FIG. 13D shows flow cytometry traces highlighting mCherry positive cell populations (representative of cells edited via the NHEJ pathway) and GFP positive cells (that underwent HDR).

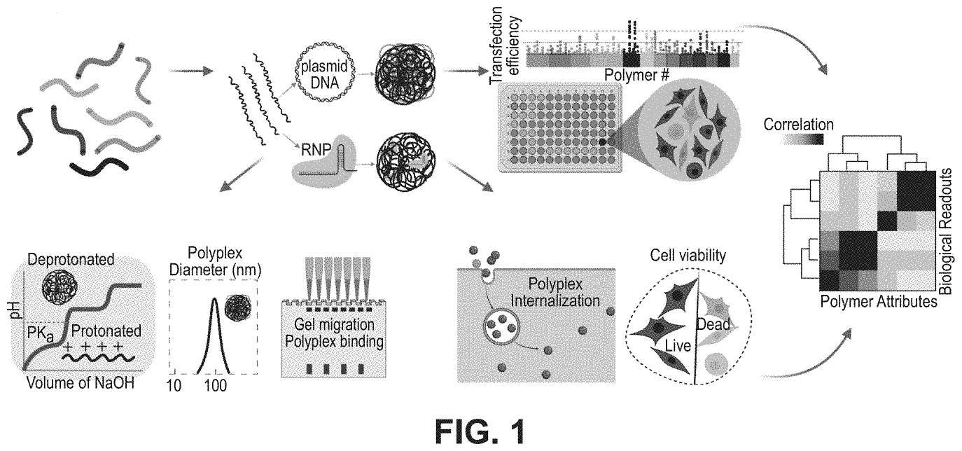

Like symbols in the drawings indicate like elements.

### DETAILED DESCRIPTION

In one aspect, the present disclosure is directed to a polymer associated with one or more biomacromolecular payloads such as, for example, pDNA, RNP, and the like to form an interpolyelectrolyte complex (also referred to herein as a polyplex). The polyplexes are internalized by a cell via various endocytic routes, the biomacromolecular payload is released inside the cell, and the payload subsequently enters into the cell nucleus to promote gene expression.

The copolymers disclosed herein thus form a well-defined complexes with biological macromolecular agents having a wide variety of chemical and biological properties, and facilitate intracellular delivery thereof. To form polyplexes and efficiently deliver the biomacromolecular cargo into a cell using, for example, a CRISPR/Cas9 delivery process, the copolymers are selected to have an array of targeted physiochemical properties such as, for example, composition, molecular weight, ζ-potential, pKa polyplex diameter, nucleic acid condensation, and combinations thereof. The copolymers and the polyplexes including the copolymers also have good gene editing efficiency, cellular internalization, cytotoxicity, and combinations thereof.

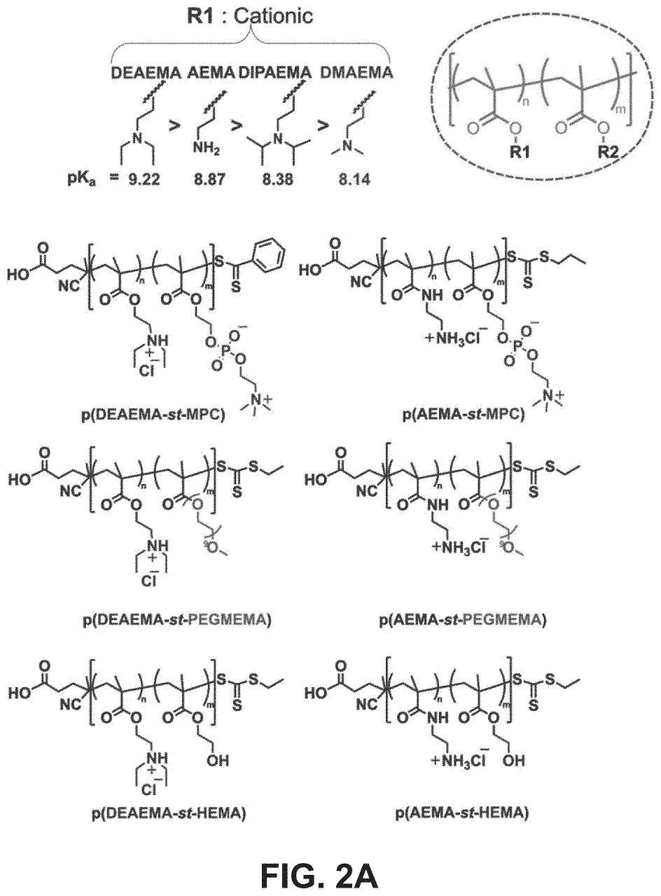

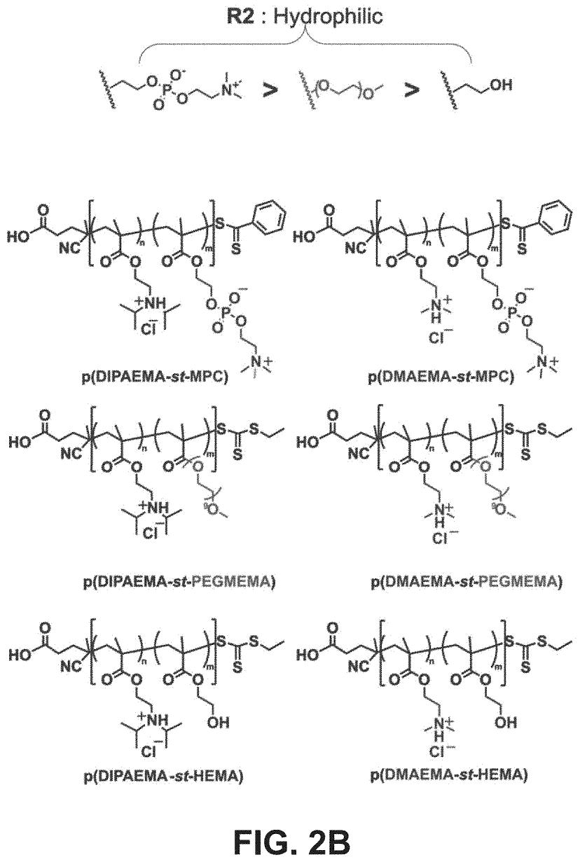

Referring to FIG. 2, the polymers of the present disclosure have a backbone including a first (meth)acryl monomeric unit with a cationic functional group R₁ and a second (meth)acryl monomeric unit with a neutral hydrophilic functional group R₂. In the present application, the term (meth)acryl refers to acryl, methacryl, acrylamido, methacrylamido, and mixtures and combinations thereof. As used herein, the term monomer, unless otherwise indicated, includes both isolated monomers and residues of monomers in an oligomer or a polymer (i.e. repeat units or residues).

In various embodiments, the cationic functional groups R₁ have amino functionality and a pKa of about 8 to about 10. In some example embodiments, the cationic functional groups R₁ include amino, alkyl amino and mixtures and combinations thereof. The alkyl groups may be linear or branched, substituted or unsubstituted, and may include cycloalkyls. In various embodiments, which are not intended to be limiting, the linear or branched alkyl groups can include 2 to 10 carbon atoms, or 3 to 7 carbon atoms, or 3 to 5 carbon atoms. The term alkylamino, which includes cycloalkylamino, as used herein, refers to an NHRp, or an NRpRq group, wherein Rₚ and R_{q} can be alkyl, or cycloalkyl.

For example, since the four cationic monomers shown in FIG. 2 vary in the type of charge center (primary vs. tertiary amines), they encompass a range of pKa values from 8-9. However, the pKa values from the resultant (co)polymers spanned a much broader range between 5.9-9 (Table 1 below). In some embodiments, in addition to amine basicity, hydrophobic interactions resulting from the structure of the amines can be important, since the alkyl substituents of the tertiary amine methacrylates vary in steric bulk and lipophilicity. In various embodiments, suitable alkyl amino groups include, but are not limited to, diethylamino, dimethylamino, diisopropylamino, and mixtures and combinations thereof.

In some example embodiments, which are not intended to be limiting, the first cationic (meth)acryl monomeric unit includes amino ethyl methacrylate (AEMA) (pKa=8.87), 2-(diethylamino) ethyl methacrylate (DEAEMA) (pKa=9.22), 2-dimethylamino ethyl methacrylate (DMAEMA) (pKa=8.14), and 2-(diisopropylamino)ethyl methacrylate (DIPAEMA) (pKa=8.38), and mixtures and combinations thereof.

While the cationic monomers including functional groups R₁ enable electrostatically mediated nucleic acid condensation, their hydrophilic reaction partners with functional groups R₂ may perform a wide variety of supporting roles such as, for example, alleviate toxicity, prolong polyplex colloidal stability, or modulate the binding equilibrium of a polyplex with a biomacromolecular compound.

Referring again to FIG. 2, in some example embodiments, which are not intended to be limiting, the hydrophilic functional groups R₂ in the polymer are neutral hydrophilic groups such as polyethylene glycol (PEG), hydroxyl (OH), phosphorylcholine, and mixtures and combinations thereof. In some embodiments, the neutral hydrophilic groups include hydroxyalkyls and alkyl phosphorylcholines. The alkyl groups may be linear or branched, substituted or unsubstituted, and may include cycloalkyls. In various embodiments, which are not intended to be limiting, the linear or branched alkyl groups can include 2 to 10 carbon atoms, or 3 to 7 carbon atoms, or 3 to 5 carbon atoms.

In some embodiments, the second neutral monomeric unit is chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof.

A library of suitable copolymers utilizing the first cationic (meth)acryl monomeric unit and the second neutral hydrophilic (meth)acryl monomeric units can be synthesized using any suitable polymerization technique such as, for example, atom transfer radical polymerization, free radical polymerization, nitroxide-mediated polymerization, reversible addition-fragmentation chain-transfer polymerization, and the like.

In the present disclosure, reversible addition-fragmentation chain-transfer polymerization (RAFT) was found to be particularly useful. In some cases, RAFT polymerization allows formation of copolymers with independent and precise control of the molecular weight distribution and composition to produce combinatorial polymer designs. For example, pairing 3 hydrophilic monomers with 4 cationic monomers creates 12 binary copolymers, and within each binary copolymer, the incorporation of cationic monomer was varied systematically to achieve compositions of 100%, 75%, 50%, 25%, and 0%.

Referring again to FIG. 2, to create the library of copolymeric candidates, the molar ratios of the monomeric units m and n were varied systematically such that cationic monomeric units (n) ranged from 0% to 100% in 25% increments, with n+m=100%. In some cases, statistical copolymers are easier to integrate within a parallel synthesis workflow, maximizing process throughput while still discovering myriad relationships between chemical structure and delivery performance.

In another embodiment, provided herein as an example, the copolymer can be synthesized in the form of a block copolymer rather than a statistical copolymer. In some embodiments, copolymers can be synthesized in two sequential steps, where the cationic monomers are first polymerized into a homopolymer and then the hydrophilic co-monomer is appended as a second block through RAFT, ATRP or NMP polymerization methods. In some embodiments, the copolymers are created as statistical copolymers through a single one-pot reaction where both the cationic and hydrophilic monomers are incorporated simultaneously.

Using the statistical technique described in the present disclosure, a library of 43 well-defined copolymers was generated using this combinatorial scheme, and the results are shown in Table 1.

TABLE 1
Characterization via NMR^{a}, SEC^{b}, titration^{c} and electrophoresis^{d}

| | | | | | M_{w}^{b} | | | ζ^{d} |
|---|---|---|---|---|---|---|---|---|
| Entry | (Co)polymer | m | n | % cat^{a} | (kDa) | D^{b} | pKₐ^{c} | (mV) |
| 1 | p(DEAEMAₘ-st-MPCₙ) | 98 | — | 100 | 19.4 | 1.05 | 6.7 | 12.4 |
| 2 | | 53 | 32 | 53 | 19.6 | 1.04 | — | −10.9 |
| 3 | | 37 | 58 | 42 | 25.4 | 1.04 | — | −14.1 |
| 4 | | 22 | 91 | 35 | 32.1 | 1.04 | — | −0.05 |
| 5 | p(AEMAₘ-st-MPCₙ) | 67 | — | 100 | 14.3 | 1.26 | 8.1 | 15.2 |
| 6 | | 50 | 23 | 68 | 19 | 1.24 | 8 | 12.8 |
| 7 | | 43 | 51 | 46 | 25 | 1.12 | 7.9 | 2.9 |
| 8 | | 19 | 61 | 24 | 22.8 | 1.05 | 6.8 | 9.9 |
| 9 | p(DIPAEMAₘ-st-MPCₙ) | 80 | — | 100 | 18.2 | 1.05 | 5.9 | 20.8 |
| 10 | | 81 | 33 | 68 | 28.2 | 1.03 | 6.4 | 5.6 |
| 11 | | 65 | 64 | 56 | 35.4 | 1.07 | 6.9 | 2.2 |
| 12 | | 22 | 91 | 29 | 32.8 | 1.02 | — | −13.8 |
| 13 | p(DMAEMAₘ-st-MPCₙ) | 110 | — | 100 | 18.4 | 1.04 | 6.9 | 21.7 |
| 14 | | 25 | 9 | 73 | 7.2 | 1.05 | 6.9 | 5.6 |
| 15 | | 48 | 32 | 60 | 17.6 | 1.01 | 7.5 | 3.8 |
| 16 | | 17 | 47 | 27 | 17.4 | 1.02 | 7.8 | 0 |
| 17 | | — | 24 | 0 | 11.3 | 1.12 | — | −10.9 |
| 18 | p(DEAEMAₘ-st-PEGMEMAₙ) | 74 | 20 | 77 | 25.6 | 1.04 | 6.9 | 4.2 |
| 19 | | 53 | 42 | 52 | 33.6 | 1.09 | 6.6 | −7.1 |
| 20 | | 36 | 65 | 33 | 43.9 | 1.06 | 6.5 | −11.7 |
| 21 | p(AEMAₘ-st-PEGMEMAₙ) | 85 | 35 | 75 | 21.8 | 1.21 | 7.8 | 16.2 |
| 22 | | 90 | 120 | 43 | 26.8 | 1.23 | 8.1 | 8.6 |
| 23 | | 44 | 79 | 26 | 25.8 | 1.19 | 7.8 | 7 |
| 24 | p(DIPAEMAₘ-st-PEGMEMAₙ) | 36 | 20 | 64 | 18.4 | 1.01 | 6.6 | 6.3 |
| 25 | | 29 | 33 | 47 | 24.4 | 1.07 | 6.8 | 2.3 |
| 26 | | 27 | 82 | 25 | 48.5 | 1.1 | 6.9 | −9.4 |
| 27 | p(DMAEMAₘ-st-PEGMEMAₙ) | 26 | 9 | 74 | 10.4 | 1.18 | 7 | 8.3 |
| 28 | | 14 | 12 | 52 | 10.6 | 1.23 | 7 | 18.5 |
| 29 | | 9 | 4 | 12 | 3.8 | 1.09 | 6.8 | −0.09 |
| 30 | | — | 7 | 0 | 6.4 | 1.77 | — | 6.8 |
| 31 | p(DEAEMAₘ-st-HEMAₙ) | 66 | 21 | 74 | 14.7 | 1.04 | 7.5 | 15.2 |
| 32 | | 47 | 47 | 50 | 16.1 | 1.07 | 7.6 | 9.4 |
| 33 | | 22 | 50 | 30 | 11.6 | 1.06 | 7.8 | 5.6 |
| 34 | p(AEMAₘ-st-HEMAₙ) | 85 | 54 | 61 | 26.2 | 1.23 | 8.2 | 22.7 |
| 35 | | 72 | 92 | 44 | 29.8 | 1.23 | 8.2 | 21 |
| 36 | | 40 | 132 | 23 | 27.1 | 1.13 | 6.9 | 18.4 |
| 37 | p(DIPAEMAₘ-st-HEMAₙ) | 61 | 33 | 65 | 19 | 1.07 | 6.5 | 16.2 |
| 38 | | 52 | 50 | 51 | 20.2 | 1.13 | 7.3 | 12.8 |
| 39 | | 27 | 80 | 25 | 17.4 | 1.07 | 6.4 | −0.7 |
| 40 | p(DMAEMAₘ-st-HEMAₙ) | 34 | 22 | 60 | 8.76 | 1.04 | 7.2 | 4.8 |
| 41 | | 32 | 45 | 42 | 11.7 | 1.05 | 7.2 | 7.9 |
| 42 | | 65 | 145 | 31 | 31.6 | 1.09 | 7.3 | 2.5 |
| 43 | | — | 60 | 0 | 8.6 | 1.04 | — | 0.8 |

To determine the most suitable copolymeric candidates for incorporation into polyplexes for transfection of biomacromolecular payloads, chemical differences among cationic moieties were considered including, but not limited to, polymer composition, molecular weight, ζ-potential, pKa, resulting polyplex diameter, and nucleic acid condensation. Resultant contrasts in polycation protonation profiles were evaluated that could potentially influence key biointerfacial processes such as payload binding, cellular internalization and intracellular release. Further, the impact of differences in co-monomer hydrophilicity and degree of incorporation were evaluated on potential impact of polyplex size, binding equilibrium with their nucleic acid payloads, and intracellular delivery.

In most cases, the composition of polymerized products mirrored that of the monomer feed, simplifying the realization of targeted cationic incorporation (100%, 75%, 50%, 25%, and 0%). Allowing RAFT reactions to reach high degrees of conversion (80-90%) modulated polymer composition merely by varying monomer feed ratios. Despite reaching high conversion, excellent control over the controlled radical polymerization process (<1.2) was achieved for almost the entire library, while also obtaining the desired degree of polymerization (a minimum Mₙ of 15 kDa was targeted). ζ-potential measurements and pKa titrations (Table 1) were performed to quantify surface potential and protonation behavior respectively of the copolymers formed. Specific details of the processes for making the copolymers are set forth in Example 1 below.

In various embodiments, a group of copolymers with particularly preferred physiochemical characteristics to form an intracellularly deliverable biomacromolecular polyplex included a first cationic methacryl monomeric unit with an alkyl amino functional group and a second neutral methacryl monomeric unit with a hydroxyl functional group. As shown in Table 1, suitable examples in the preferred group include, but are not limited to, p(DEAEMAₘ-st-HEMAₙ), p(AEMAₘ-st-HEMAₙ), p(DIPAEMAₘ-st-HEMAₙ), and p(DMAEMAₘ-st-HEMAₙ). These copolymers were selected by a number of characterization test results, which are briefly outlined in the discussion below. More specific details of the polymer formation and characterization procedures are set forth in Example 1 below.

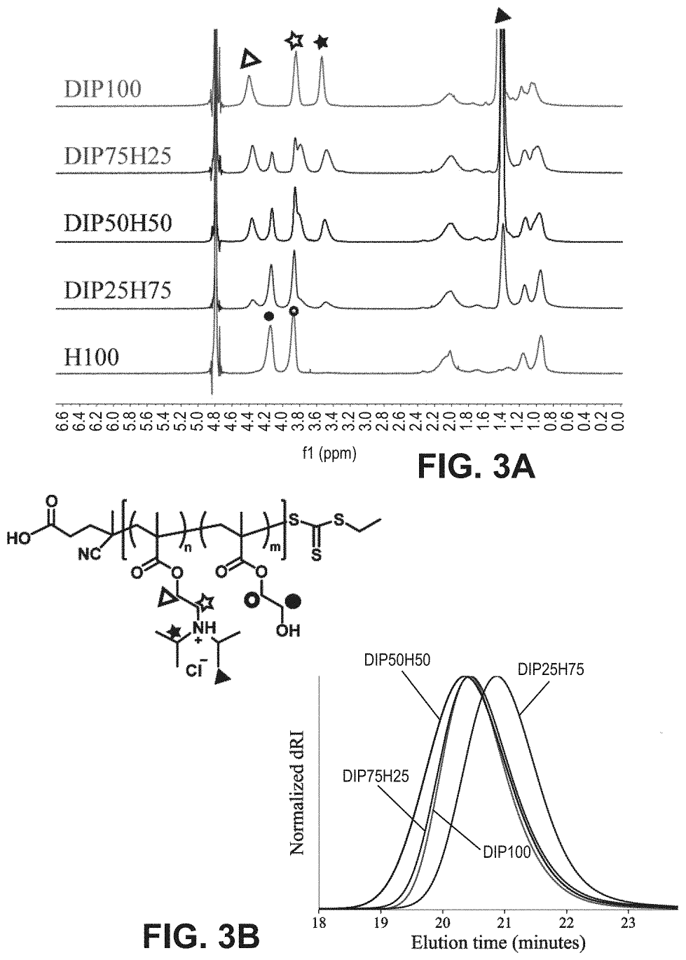

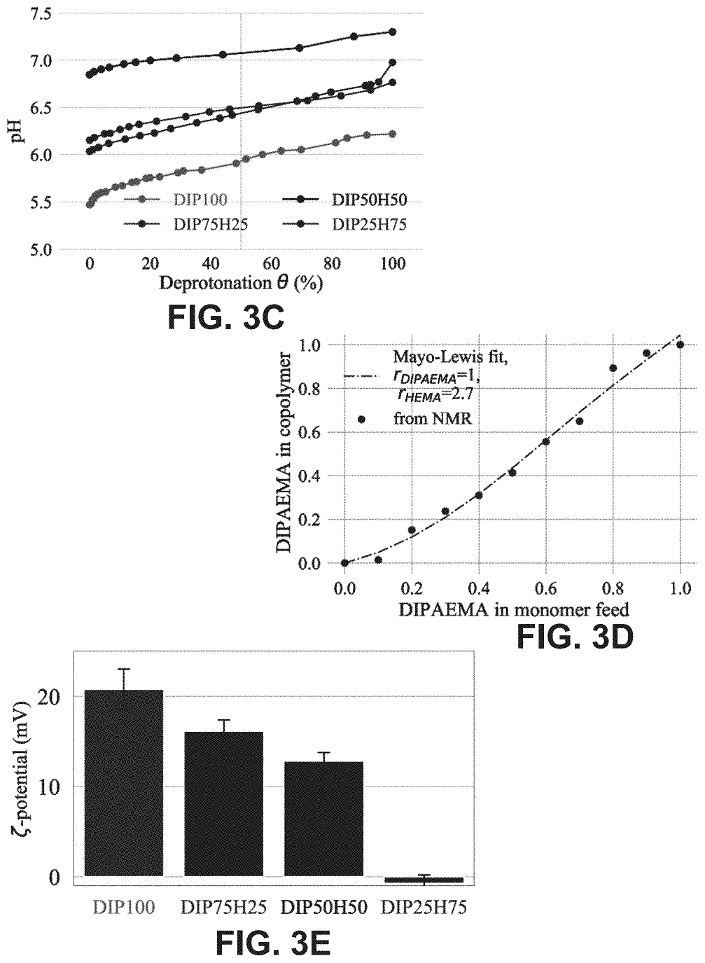

Proton NMR of purified the copolymers DEAEMA, AEMA, DIPAEMA, and DMAEMA of Table 1 indicated that peaks representative of HEMA and DIPAEMA pendant chains occur along distinct regions, which allowed for a relatively simple computation of polymer composition (FIG. 3A). NMR results were obtained on a suitable instrument such as, for example, those available under the trade designation Avance III HD 500, from Bruker, Billerica, Mass.

As shown in Table 1, all four cationic polymers DEAEMA, AEMA, DIPAEMA, and DMAEMA as a group had a M_{w} of about 5 to about 35 kDa. Molecular weight can be determined by any suitable technique, and size exclusion chromatography (SEC) using equipment and reagents available from Agilent Technologies, Santa Clara, Calif. was performed with refractive index and multiple angle light scattering detectors (Wyatt, Santa Barbara, Calif.) to determine the complete molecular weight distribution for all copolymers. For all four cationic polymers DEAEMA, AEMA, DIPAEMA, and DMAEMA, SEC traces revealed monomodal populations of polymers with low dispersities, suggesting that polymerization followed a trajectory defined by RAFT kinetics (FIG. 3B).

The effect of copolymer composition on deprotonation was determined by estimating pKa values via titration using, for example, a pH titrator available from ThermoFisher Scientific, Waltham, Mass., under the trade designation Orion Star T901. Compared to the cationic homopolymer DIP100, the copolymers exhibited higher pKa values, which highlighted the impact of polymer composition on polycation protonation (FIG. 3C). As shown in Table 1, the copolymers DEAEMA, AEMA, DIPAEMA, and DMAEMA as a group had a pKa of about 6.0 to about 8.5.

In addition, reactivity ratios of the copolymers constituted from DIPAEMA and HEMA were determined using the Mayo-Lewis model to visualize the probable monomer sequences. (FIG. 3D). The results indicated that HEMA dominated the initial phase of polymerization, while the two monomers co-polymerized in statistical fashion in the middle phase. The results further indicated that once HEMA was nearly consumed, the ends of the polymer chains contained a high frequency of DIPAEMA repeat units.

As shown in FIG. 3E, ζ-potential measurements of the copolymers DEAEMA, AEMA, DIPAEMA, and DMAEMA in phosphate buffered saline (PBS) revealed that while the cationic homopolymer displayed the highest surface charge, the addition of HEMA repeat units gradually lowered the ζ-potential. Referring again to Table 1, the ζ-potential measurements of the copolymers DEAEMA, AEMA, DIPAEMA, and DMAEMA revealed that while the cationic homopolymer displayed the highest surface charge, the addition of HEMA repeat units gradually lowered the ζ-potential. As shown in Table 1, the copolymers DEAEMA, AEMA, DIPAEMA, and DMAEMA as a group had a ζ-potential of about −1 mV to about 25 mV. The Malvern Zetasizer (Malvern Instruments, MA) was used to evaluate the ζ-potential of all polymers in the library through electrophoresis.

Considering all the above parameters, one particularly useful copolymer from the selected group in the library was determined to be p(DIPAEMAₘ-st-HEMAₙ), which is shown in FIG. 2 and Table 1. As shown in Table 1, these copolymers included p(DIPAEMA₆₁-st-HEMA₃₃), p(DIPAEMA₅₂-st-HEMA₅₀), and p(DIPAEMA₂₇-st-HEMA₈₀). As a group, these copolymers had a M_{w} of about 15 kDa to about 25 kDa, or about 17 kDa to about 20 kDa, a pKa of about 6.4 to about 7.3, and a ζ-potential of about −1 mV to about 20 mV, or about −1 mV to about 16 mV. Of this group of copolymers, p(DIPAEMA₅₂-st-HEMA₅₀) was found to have a particularly useful combination of the measured properties.

Having obtained polymers with the desired lengths and compositions, copolymeric structures were identified that mediated efficient genome editing, with a view toward complexing the copolymers with biomacromolecular payloads to form polyplexes for intracellular delivery. As part of this identification process, an engineered HEK293 cell line was used that expressed the traffic light reporter (TLR) gene. The TLR system resolves and quantifies two possible pathways of gene editing, one of which will be activated following the site-specific cleavage of DNA by Cas9.

The first pathway included non-homologous end-joining (NHEJ), an imprecise and potentially error prone DNA-repair event that produces several frameshift mutations, a third of which contribute to translation of an mCherry coding region.

The second pathway included precise insertion of repair templates, either single-stranded oligonucleotides (ssODN) or pDNA, at the site of the double-stranded break that would lead to GFP production through homology-directed repair (HDR). In some embodiments suitable copolymers should function as effective vehicles for both ribonucleoproteins (RNP) as well as pDNA.

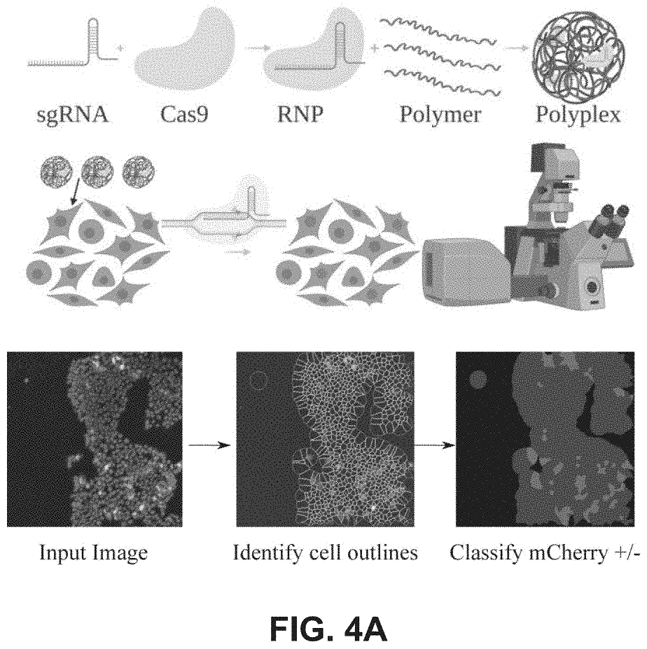

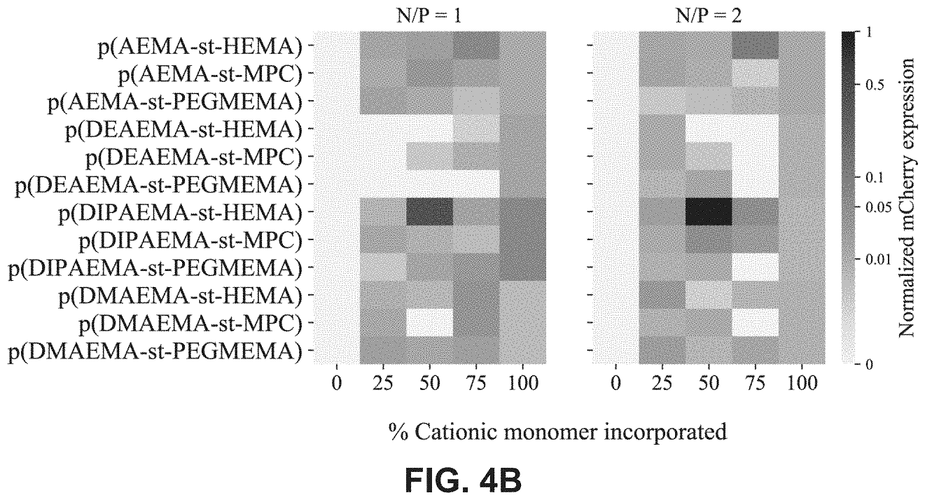

To rapidly interrogate NHEJ events resulting from various copolymeric vectors in the library, a robust high-throughput assay was used to quantify mCherry expression in transfected cells. Rapid quantification of NHEJ editing, while ensuring adequate sensitivity to low gene editing efficiencies was challenging, given the constraints imposed by the TLR cell line. Even if 100% NHEJ editing were to be achieved, in some embodiments only 33% of frameshift mutations would cause the mCherry fluorescent protein to be expressed, hindering the detection and quantification of low editing efficiency. To resolve the trade-off between throughput and sensitivity, image cytometry was used, which in some embodiments can be an excellent alternative to more tedious approaches such as flow cytometry (FIG. 4A).

As shown in FIG. 4A, during screening studies, HEK293 TLR cells were cultured in 48-well plates and 86 distinct polyplex formulations were evaluated (43 polymers at N/P ratios of 1 and 2). Forty-eight hours after transfection, cells were inspected through a microscope equipped with automated image acquisition features, and 3-5 fields of view were captured per well, yielding 6-10 images per treatment condition. To analyze NHEJ events in an automated, rapid and accurate manner, mCherry expression from fluorescence images was quantified using a custom-designed image processing pipeline.

To compare the incidence of NHEJ as a function of polymer composition and N/P ratios (FIG. 4B), for each polyplex formulation, mCherry expression levels were averaged over multiple fields of view and subsequently normalized to the maximum mCherry expression recorded across the polymer library. In the example embodiment described in FIG. 4B, the copolymer p(DIPAEMA₅₂-st-HEMA₅₀), also referred to herein generally as DIP50H50 or DIPAEMA-HEMA, exhibited the highest levels of NHEJ editing at both N/P ratios, which suggested that polyplexes made with this copolymer mediated efficient intracellular RNP delivery.

Excepting DIP50H50, the rest of the copolymers in the library fell into two classes: either near-zero mCherry expression or marginal mCherry expression (<0.25) levels were recorded. While not wishing to be bound by any theory, such a vast gap separating DIP50H50 from second-tier formulations may indicate an “all-or-nothing” bimodal pattern in the RNP delivery data, but further study via statistical learning tools is required for confirmation. The extremely low hit rate (1/43) and the absence of clear-cut relationships between transfection efficiency and vehicular features such as chemical composition and formulation ratio, illustrated the power of the high-throughput approach, since DIP50H50 would have likely not been discovered through a traditional hypothesis-driven approach.

Having identified DIP50H50 as a copolymer that mediated efficient RNP delivery, precise HDR-mediated gene insertion was evaluated, which included co-delivery of DNA repair templates in the form of pDNA, along with the RNP payloads. Several therapeutic applications, such as engineering T-cells to recognize tumor-associated antigens and initiate anti-tumoral responses, can require challenging genomic modifications wherein extremely large DNA sequences (>1 kb) must be inserted via homology-directed repair (HDR). Despite the evolution of cutting-edge gene tools such as base editing and prime editing, gene repair through HDR retains strong therapeutic relevance since in some embodiments it can be the most efficient way to replace a certain gene variant with another in an error-free manner. Packaging and delivering both payloads within a single polyplex is expected to promote spatial and temporal co-localization of both sets of editing machinery, shifting the odds in favor of the HDR pathway. If the design rules underlying polymeric delivery of plasmid DNA overlap with those for RNP payloads, the polymer identified during the RNP delivery screening would more likely suffice for the donor.

To verify this assumption, a second screening campaign was conducted in which a model pDNA (pZsgreen) was used that transiently enhances GFP expression. While pZsgreen induces transient GFP expression, the donor pDNA permanently inserts a GFP-coding region at the site of the double-stranded break. Despite differences in biological function, pZsgreen and the donor pDNA resemble each other in their surface charge, size and binding affinity to the polymer. The screening campaign assumed that screening experiments employing the model plasmid, pZsgreen would be predictive of polymer interactions with the HDR donor pDNA.

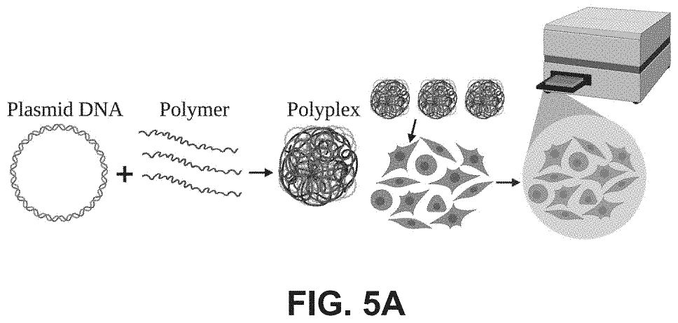

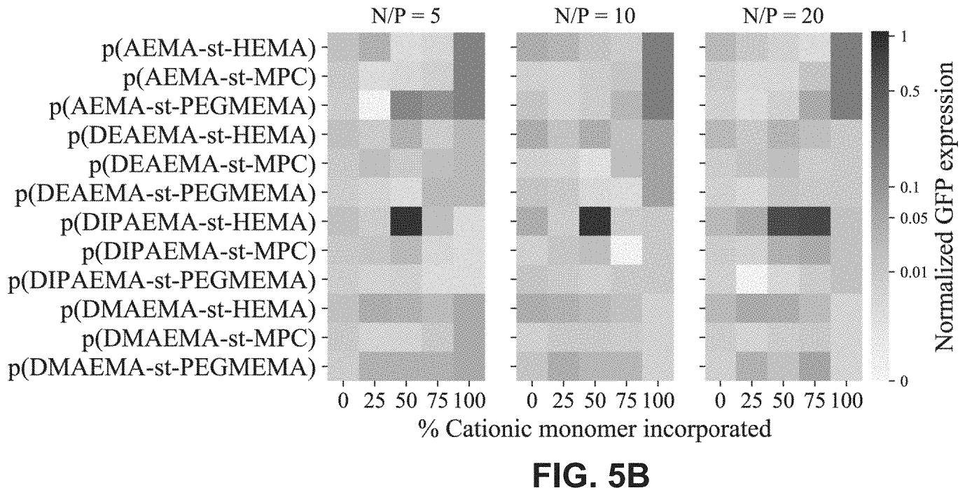

During transient transfection screening studies, cells were transfected with 129 distinct formulations, arising from the complexation of pZsgreen with 43 distinct polymers at three N/P ratios (5, 10, and 20). GFP production in transfected cells was measured using a standard plate reader and the average reading for each treatment group was normalized to that obtained from Lipofectamine 2000, a lipid-based reagent for pDNA delivery (FIG. 5B). DIP50H50 was also the best functioning copolymeric vehicle for pDNA payloads. While DIP50H50 polyplexes resulted in the highest GFP readings, high levels of GFP expression were also detected in cells treated with a closely related analog, DIP75H25 (at an N/P value of 20).

Using data science approaches, the screening outcomes for pDNA payloads were compared with those for RNPs, resulting in the conclusion that the structural drivers for plasmid delivery and RNP delivery overlap to great extent.

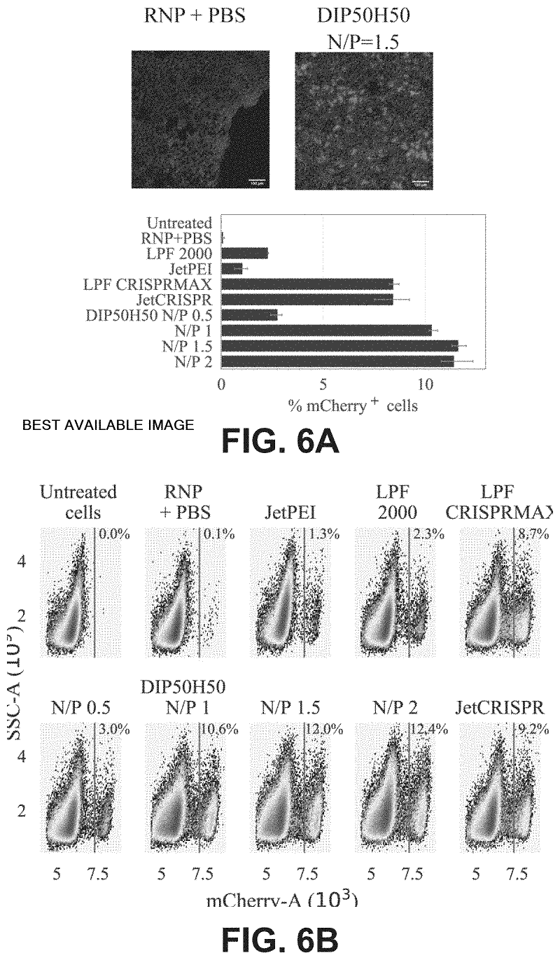

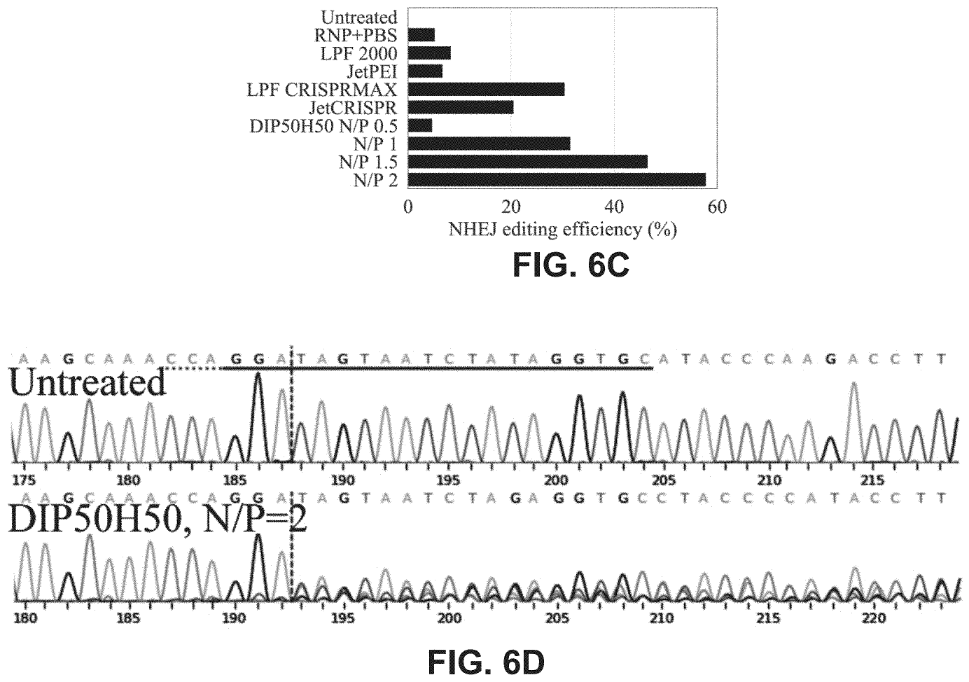

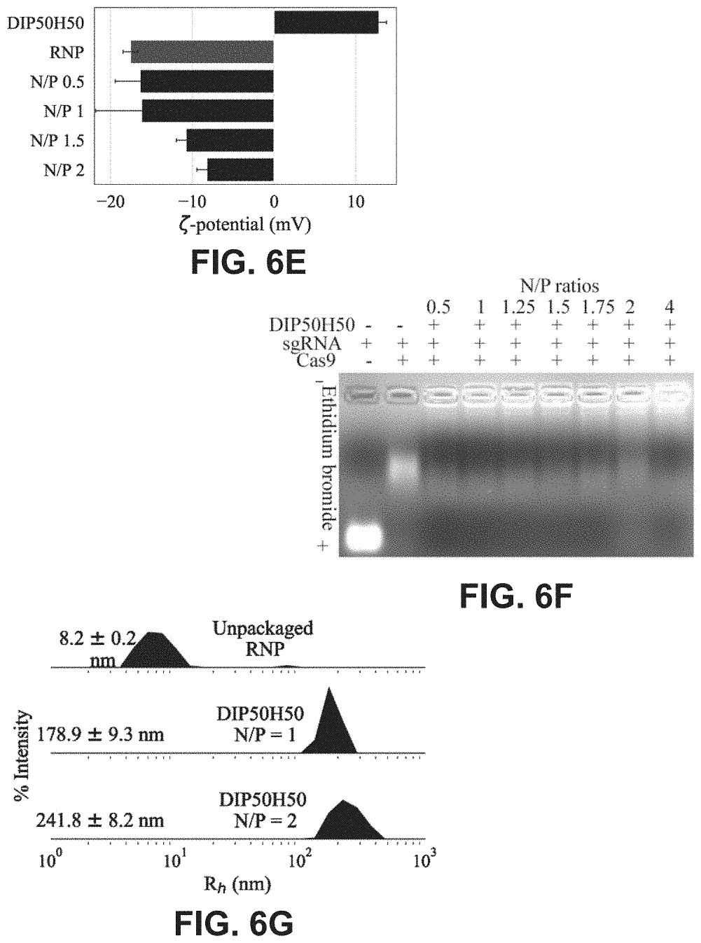

The copolymer DIP50H50 was also benchmarked against commercially available lipid and PEI-based transfection using flow cytometry (FIG. 6A). As shown in FIG. 6A, RNP transfection by JetPEI and Lipofectamine 2000 resulted in 1% and 2% mCherry expression respectively, suggesting that these reagents are better suited for pDNA payloads than for RNPs (FIG. 6B). The RNP specific reagents fared better, with both Lipofectamine CRISPRMAX and JetCRISPR causing around 8% of transfected cells to express mCherry.

As for DIP50H50, in some embodiments studied mCherry expression was highly dependent on the dose of polymer selected. In some cases, at an N/P of 0.5, editing performance was marginal, with only 2% mCherry expression, however N/P ratios of 1, 1.5 and 2 resulted in significant improvements over commercial controls, with 10-12% of transfected cells producing mCherry (FIG. 6B).

In the TLR cell line, in some cases mCherry expression can severely underestimate the actual editing efficiency, since only a fraction of gene disruptions may result in mCherry production. Flow cytometry was performed with an analysis of the distribution of insertions and deletions (indels) culminating from NHEJ (FIG. 6C). Sequencing results largely mirrored the trends observed during flow cytometric measurements, establishing that in some cases mCherry expression is a valid proxy for editing efficiency. Among commercial reagents, the highest editing efficiency was determined for Lipofectamine CRISPRMAX, with 30% of the cell population containing mixed DNA sequences resulting from error-prone NHEJ.

In the embodiment of FIG. 6, JetCRISPR reported slightly lower editing frequencies than expected (20%) while less than 10% indel formation was observed for JetPEI and Lipofectamine 2000. In contrast, DIP50H50-mediated RNP delivery led to editing efficiencies as high as 58% at an N/P ratio of 2, which is almost double that of Lipofectamine CRISPRMAX. In the example of FIG. 6, the editing efficiency of DIP50H50 spanned a broad range between 2-58%, emphasizing the strong influence of N/P ratio. Taken together, flow cytometry and sequencing results can provide evidence that a preferred copolymer such as DIP50H50 outperforms state-of-the-art commercial vehicles.

To identify the biophysical factors associated with functional RNP delivery, gel migration studies and dynamic light scattering (DLS) measurements were performed for the entire polyplex library. Since polymeric vectors should balance payload protection and rapid intracellular unpackaging, binding states were probed using simple gel migration assays. In the case of DIP50H50, RNPs existed in two states: polymer-bound RNPs that remained immobile as well as a small population of RNPs that migrated (FIG. 6F). To gain further insight into polymer-RNP association, ζ-potential measurements of polyplexes were performed (FIG. 6E). The spCas9 protein is known to possess an electrostatically heterogeneous surface, while the sgRNA has an anionic backbone. As a result, RNP complex bears a charge of −17.5 mV in PBS.

Among polyplex formulations with N/P ratios of 0.5 and 1, the extent of negative charge was reduced, but polarity was not reversed. Surprisingly, charge inversion was not achieved even at higher N/P ratios of 1.5 and 2, although the negative charge was reduced in magnitude. The unexpected observation of net negative charge may be explained by the presence of free RNPs co-existing with weakly bound RNP-polymer complexes, mirroring findings from gel migration. Given the success of DIP50H50, in some embodiments it appears that the formation of tightly bound polymer-RNP complexes is not essential for efficient delivery.

RNPs formulated with DIP50H50 at N/P ratios of 1 and 2 were around 180 nm and 240 nm in hydrodynamic radius, which is around 20 times the size of the unpackaged RNP (FIG. 6G).

In some embodiments, the tendency of the copolymer to form polyplexes larger than 100 nm in size can have implications for cellular internalization and nuclear accumulation. While particles smaller than 100 nm in radius exhibit a preference for clathrin-mediated pathways, larger particles are internalized via caveolar pathways, which permit polyplexes to traverse the cytosol and enter the nucleus while avoiding lysosomal interrogation. In addition, physical aspects of in vitro transfection cannot be overlooked: upon introduction into the cell culture media, bulkier polyplexes will reach the surface of adherent monolayer cultures faster than smaller polyplexes. The large size of DIP50H50 polyplexes may have eliminated the need to improvise endosomal escape routes and imparted favorable transport characteristics that maximized polyplex-cell contact.

Co-monomer hydrophilicity can also in some cases have an impact on polyplex diameter as well as RNP-binding affinity. While HEMA-based copolymers tended to form large assemblies, (Rh of 200-600 nm), MPC and PEG copolymers consistently formed RNP-sized polyplexes smaller than 10 nm. Aggregation was a frequent occurrence in HEMA-based copolymers while the original size distribution of unbound RNPs was preserved when hydrophilic structural motifs such as PEG and MPC were present. In the embodiment of FIG. 6, none of the hydrophilic polymers incorporating MPC and PEG retained RNP payloads, while some AEMA-based copolymers exhibited complete retention of RNP payloads.

The preferred copolymer discovered through this combinatorial screen does not confirm to traditional heuristics described in the literature. In one aspect, binding between the RNP payload and the polymer was incomplete, with a small fraction of RNPs remaining unbound. In another aspect, since polyplexes are negatively charged, electrostatic interactions did not contribute significantly to efficient intracellular delivery. In another aspect, the polyplex diameter exceeded 200 nm, and yet these particles are quite adept in evading endosomal capture to enter the nucleus. Despite these departures from the expected polymeric vector design rules, the hit polymer DIP50H50 proved to be an excellent chemical vector with a transfection efficiency far higher than that of commercially available reagents,

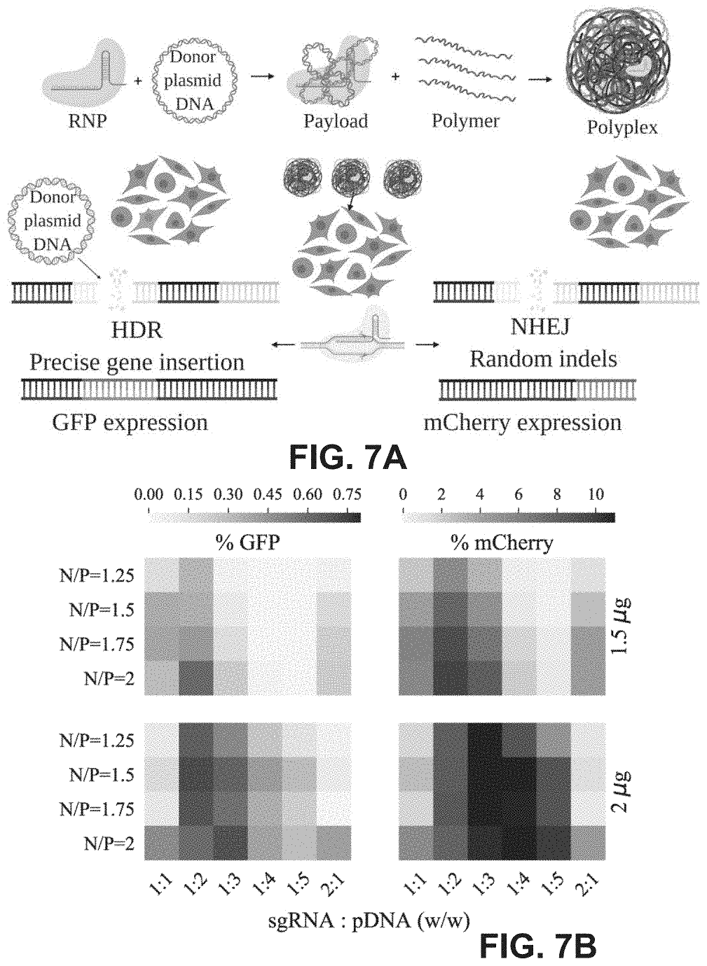

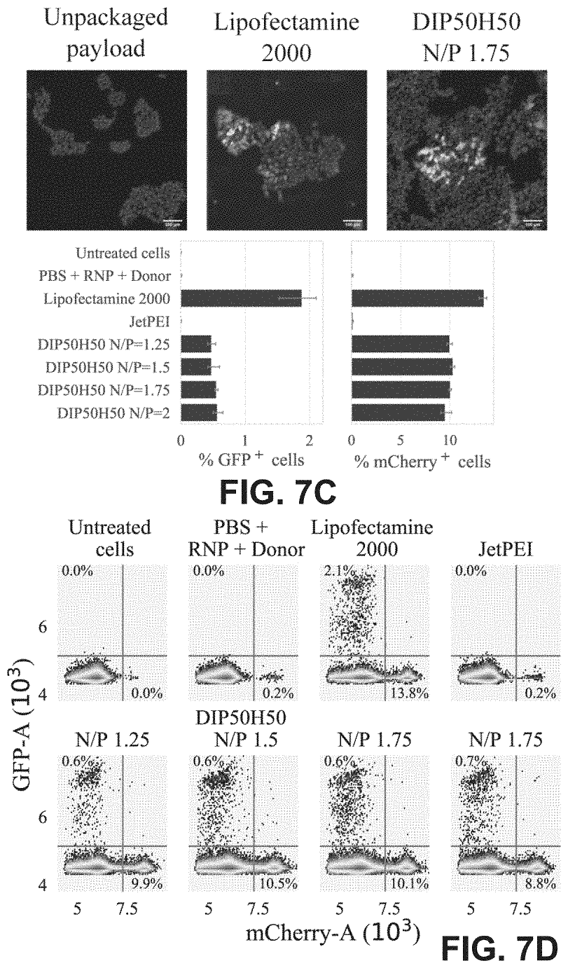

Rational design of polyplexes for faithful DNA repair via HDR pathways requires optimization of three variables: the total nucleic acid dose, the proportion of sgRNA relative to the donor pDNA, and the polymer loading or N/P ratio. Factorial experiment design was used to simultaneously examine the effects of 1) the total nucleic acid dose, which was studied at 1.5 and 2 μg levels, 2) payload composition or the weight ratio of sgRNA to pDNA (w/w ratios of 2:1, 1:1, 1:2, 1:3, 1:4 and 1:5) and 3) N/P ratio (1,1.25,1.5,2). The payload composition was varied while keeping the total nucleic acid dose fixed at 1.5 or 2 μg per well for a 24-well plate. Taken together, 48 conditions were evaluated in this experimental matrix, allowing us to discover the optimal conditions for HDR editing (FIG. 7B).

The relative frequencies of NHEJ and HDR was quantified by measuring mCherry and GFP expression, respectively. From flow cytometric measurements showed that both the rate of integration of the donor plasmid (quantified via GFP readouts) as well as the formation of random indels (measured via mCherry expression) were highest when the nucleic acid loading was maximum (2 μg/well for a 24-well plate). A non-monotonic relationship was determined between HDR frequency and the payload composition, where both sgRNA-dominant payloads (2:1) and pDNA-dominant payloads (1:5) conditions resulted in extremely low HDR frequencies (<0.1%) while intermediate payload compositions (1:2 and 1:3 w/w mixtures) resulted in the highest GFP expression (0.7%). In some cases, mCherry expression was also highest at intermediate compositions, which can suggest that both RNP and pDNA incorporation within polyplexes is highest at this mixing ratio.

The HDR performance of the hit polymer DIP50H50 was then benchmarked against commercial controls at the optimized polyplex formation conditions of 2 μg nucleic acid dose composed of a 1:2 w/w ratio of sgRNA and donor pDNA. Both mCherry and GFP expression were measured, indicative of NHEJ and HDR editing respectively, in cells treated with DIP50H50 at N/P ratios of 1.25, 1.5, 1.75 and 2. Lipofectamine 2000 and JetPEI were also included as positive controls (FIG. 7D).

While JetPEI resulted in scarcely any HDR-edited cells, Lipofectamine 2000 was the only reagent where more than 2% of the cell population was GFP-positive. GFP expression did not exceed 0.7% when DIP50HEMA50 polymers were used to deliver HDR constructs, consistent with the results observed during the payload optimization experiment.

While not wishing to be bound by any theory, the evidence indicated that the causes underlying low HDR frequencies originate in cellular processes rather than polymeric design. Transfection was not synchronized with cell cycle, nor were HDR-promoting drugs used to bias editing in favor of gene insertion. Despite the absence of biological and chemical intervention, a substantial pool of cells were obtained that underwent the HDR editing pathway, a cell population that can subsequently be sorted and expanded to fulfill therapeutic demands. Having demonstrated the viability of the preferred copolymer for HDR applications in these proof-of-concept studies, the focus was shifted from co-delivery of separate untethered RNP and donor payload to tethered payloads.

The systematic screening strategy as described in the present disclosure, aided by high-throughput experimentation and combinatorial design rapidly yielded the promising lead structure (DIP50H50). Yet, the low hit rate (1/43) observed during the screening campaign described above and the lack of obvious structural drivers of DIP50H50's intracellular delivery efficacy prompted review using the data science techniques in Example 2 below.

The polymeric carriers of the present disclosure mediate efficient RNP delivery, but the present disclosure is further directed to additional embodiments in homology-directed repair (HDR)-mediated gene insertion, which requires co-delivery of pDNA repair templates along with the RNP payloads. Several therapeutic applications, such as engineering T-cells to recognize tumor-associated antigens and initiate anti-tumoral responses, require challenging genomic modifications wherein extremely large DNA sequences (>1 kb) must be inserted via HDR. Despite the evolution of cutting-edge gene tools such as base editing and prime editing, gene repair through HDR retains strong therapeutic relevance since it is the best way to replace a certain gene variant with another in an error-free manner. Packaging and delivering both payloads within a single polyplex is expected to promote spatial and temporal co-localization of both sets of editing machinery, shifting the odds in favor of the HDR pathway. However, this is a challenging goal since RNPs and pDNA possess distinct molecular attributes, right from their size (193 kDa vs. 3 MDa), charge (−17.5 mV vs. −40 mV), surface chemistry, and binding affinities.

HDR requires the insertion of a pDNA template at the site of the double stranded break to achieve precise genomic corrections. To resolve and quantify gene editing pathways, HEK293 cells were engineered with the TLR gene, wherein mCherry is expressed if imprecise editing, characterized by random indels, is dominant. Whereas, if the GFP-coding donor pDNA template is successfully inserted through the HDR pathway, GFP would be expressed instead.

A model pDNA (pZsgreen) that transiently enhances GFP expression within transfected cells was employed that induces transient GFP expression, and the donor pDNA permanently inserts a GFP-coding region at the site of the double-stranded break. Despite differences in biological function, pZsgreen and the donor pDNA are very similar in their surface charge, size and binding affinities to polycations, making pZsgreen an excellent model for the donor DNA. In screening experiments employing the model plasmid, pZsgreen, was considered predictive of polymer interactions with the HDR donor pDNA. Although, no genomic editing occurs during transient transfection, this screening study helped discover polymers that were suitable for delivering pDNA payloads during HDR.

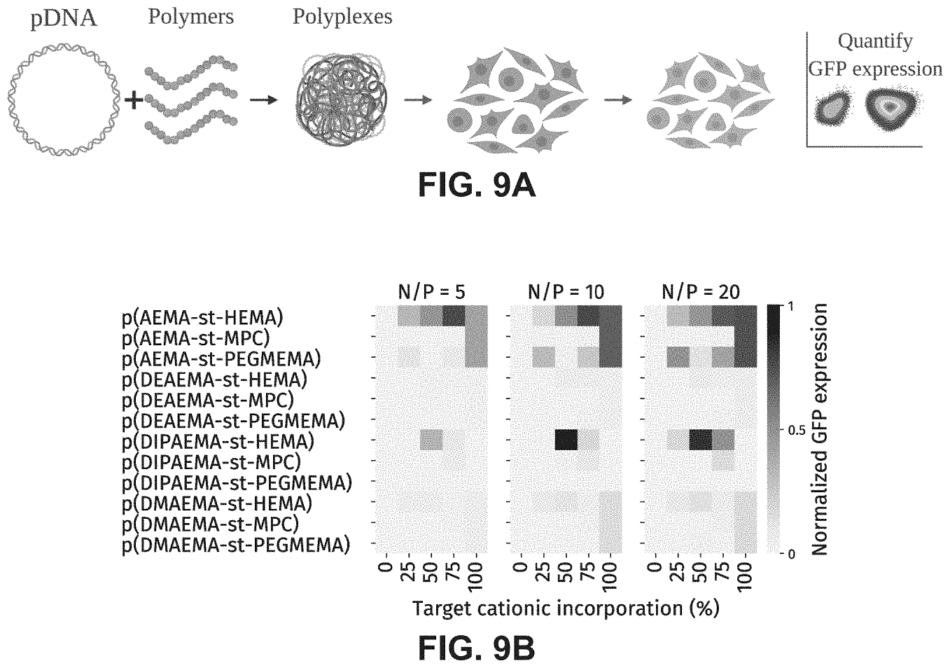

As shown in the schematic representation in FIG. 9A, HEK293 cells were transfected with 129 distinct formulations, arising from the complexation of pZsgreen with the 43 distinct polymers of Example 1 below at three N/P ratios (5, 10, and 20). GFP production in transfected cells was measured using flow cytometry and the proportion of GFP-expressing cells calculated (FIG. 9B). The percentage uptake across the library was normalized to the highest value in the library and plotted as a geat map.

DIP50H50, the hit polymer identified above as also being most useful for RNP delivery, resulted in the highest GFP readings among the entire polymer library. While DIP50H50 was undoubtedly the top-performer, high levels of GFP expression were also detected in cells treated with a closely related analog, DIP75H25, and with several polymers constituted from AEMA.

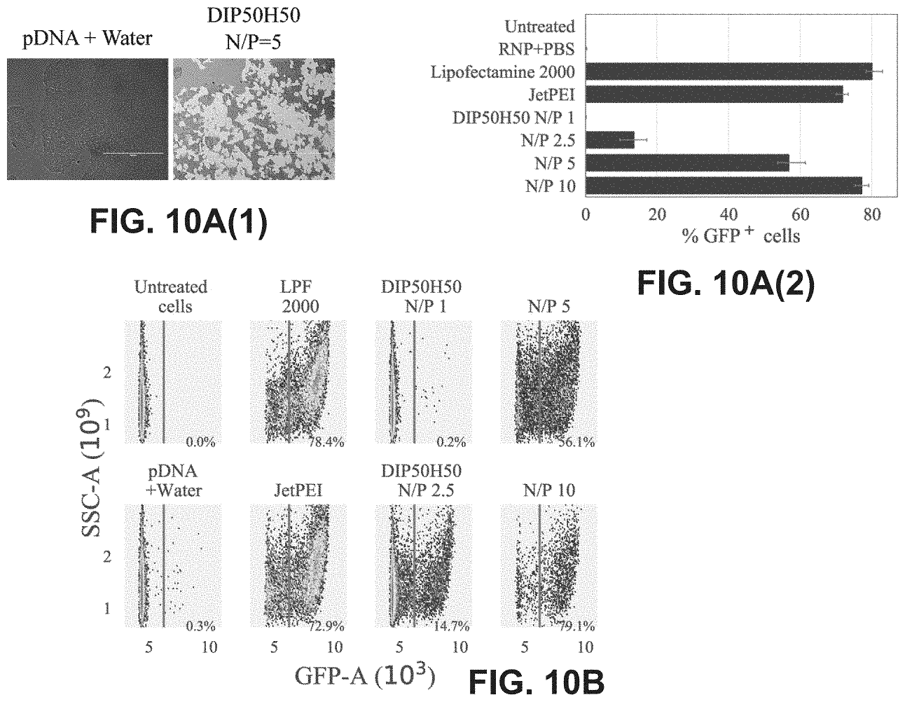

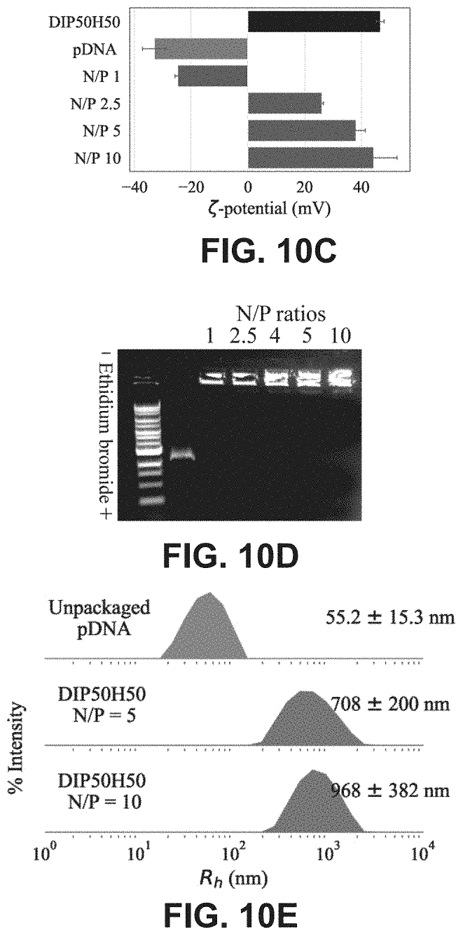

The pDNA delivery functionality of DI50H50 was benchmarked against commercial reagents specially developed for pDNA transfection, Lipofectamine 2000 and JetPEI (FIGS. 10A-10E). Thereafter, GFP production in transiently transfected cells was measured using flow cytometry. Unlike the case of RNP delivery, both JetPEI and LPF 2000 achieved efficient pDNA delivery and promoted GFP expression in more than 90% of the cell population. No GFP was detected when the hit polymer DIP50H50 was employed at an N/P of 1, but GFP expression levels improved at higher N/P ratios, climbing to 25% at an N/P of 2.5 and about 80% at N/P ratios of 5 and 10. Polyplexes formulated at an N/P of 10 outperformed JetPEI (p=0.01, t-test) and were comparable to Lipofectamine 2000 (no significant differences) while delivering pDNA. The hit polymer DIP50H50 proved to be an effective vector for not only RNP payloads to mediate NHEJ editing, but also for pDNA to promote transient transfection.

Given that transfection efficiency was N/P-dependent, with extremely low efficiencies observed at N/P ratios of 1 and 2.5, gel migration assays were performed to study pDNA-polymer complexation as a function of N/P ratios. While unpackaged pDNA was able to migrate unhindered, the hit polymer retarded pDNA mobility at all N/P ratios studied, even at the lowest N/P of 1. The data from gel migration showed that that the polymer-plasmid binding was sufficiently strong enough to package and protect pDNA during delivery.

To further probe N/P dependence, ζ-potential measurements were conducted and obtained values ranging from −40 mV for unpackaged plasmid to −25 mV for the N/P=1 formulation to 50 mV for N/P ratios of 2.5 and above. A neutral surface charge was not observed for the N/P=1 formulation, suggesting that the negative surface potential of N/P=1 formulations may have inhibited membrane association and subsequent cellular uptake. However, at an N/P ratio of 2.5, pDNA was not only neutralized, but polyplexes also a acquired positively charge, the magnitude of which grew significantly when more DIP50H50 was added to achieve higher N/P ratios of 5 and 10.

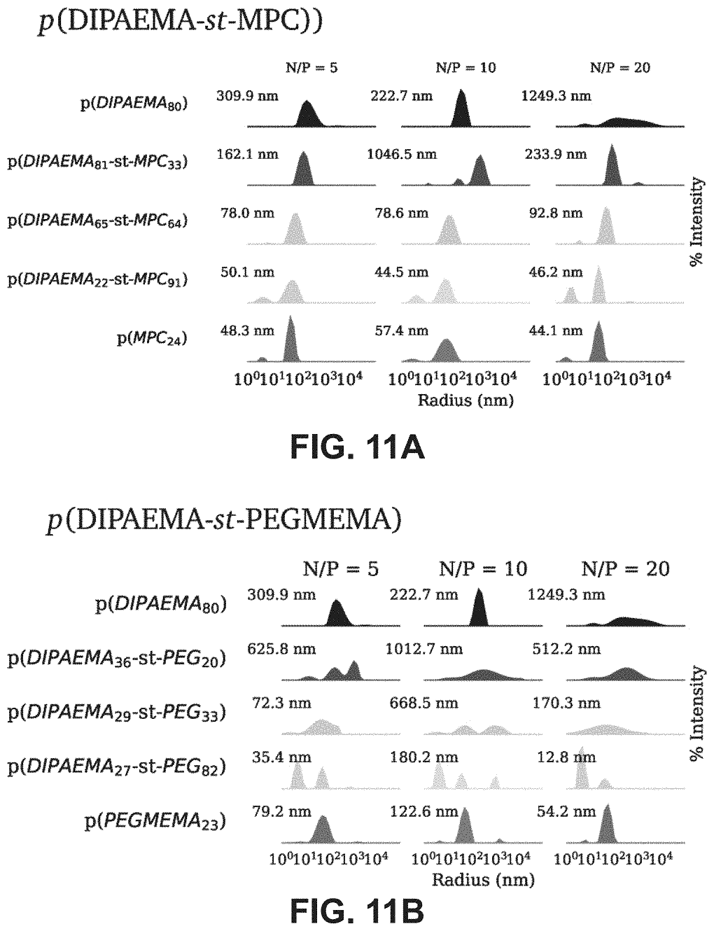

Further, the effects of polymer composition and N/P ratio on polyplex size distributions was systematically examined using dynamic light scattering (DLS), and observed that polymer hydrophilicity and polyplex diameter were negatively correlated. While unimodal populations with diameters approaching 1 μm in radius were formed when DIP50HEMA50 was complexed with pDNA, polyplex sizes were greatly reduced when PEGMA or MPC co-monomers were employed in the place of HEMA (FIGS. 11A-11B).

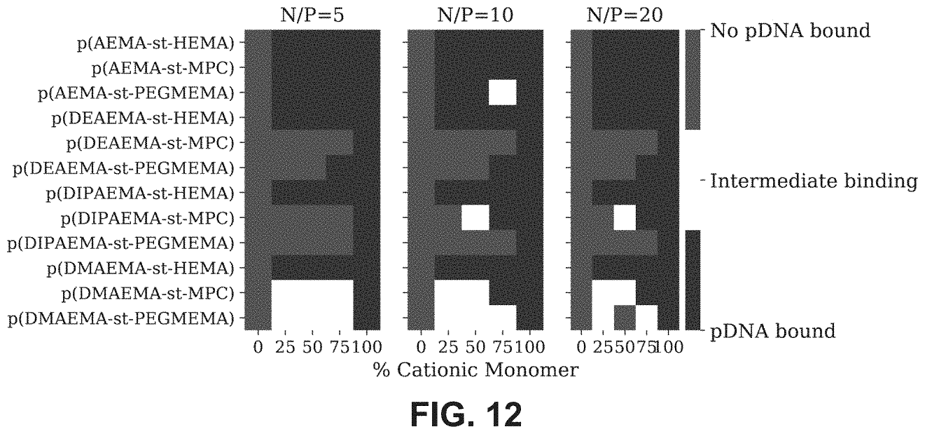

In these hydrophilic variants of DIP50H50, two distinct populations were discerned: the free polymer and the unbound plasmids, suggesting that highly hydrophilic co-monomers inhibit polymer-plasmid binding. Indeed, gel migration assays that quantified the strength of pDNA-polymer association, confirmed the existence of two sub-populations for these hydrophilic variants, offering further evidence that highly hydrophilic co-monomers inhibit polymer-plasmid binding. Most PEG and MPC copolymers failed to retain pDNA payloads during gel migration assays and did not form tightly condensed polyplexes in contrast to the strong binding displayed by HEMA-based copolymers (FIG. 12) Additionally, polyplex diameters appears to be reduced when the incorporation of PEGMA or MPC was increased for almost all polymers in the library.

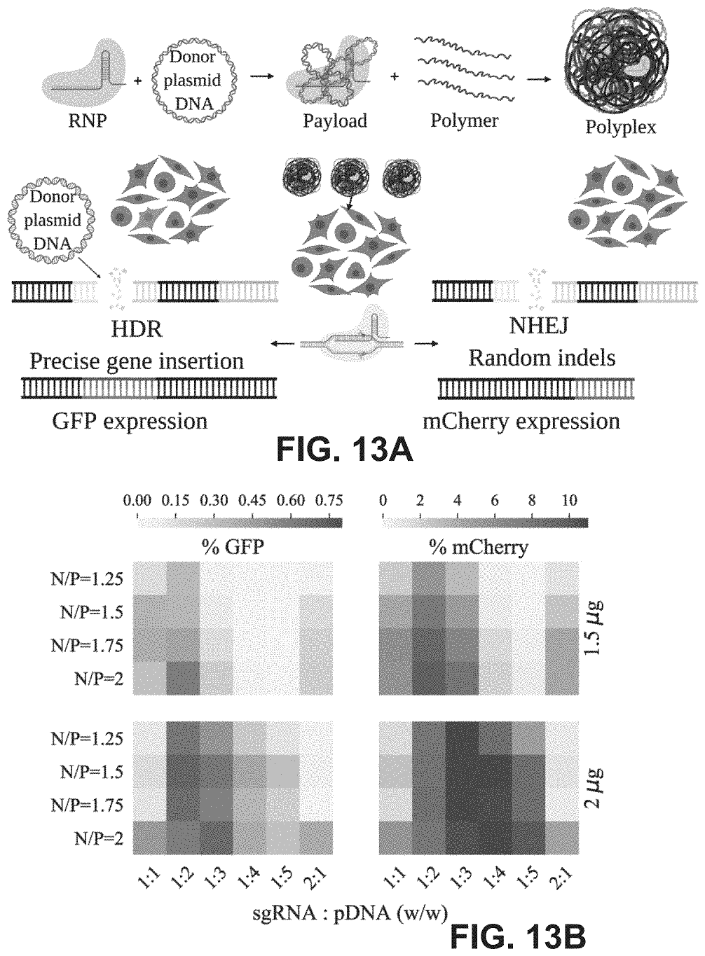

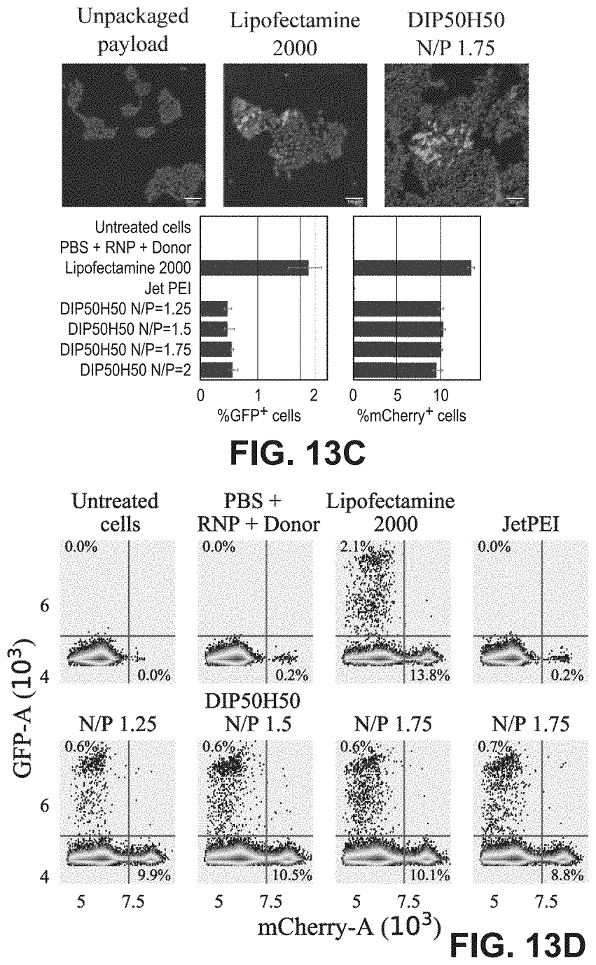

As shown schematically in FIG. 13A, having identified a polymer that could deliver both RNP payloads during NHEJ editing and for pDNA during transient transfection, co-delivering RNP with a pDNA donor to effect precise gene knock-in via HDR editing was investigated. Molecular pathways favoring HDR events are activated when donor DNA, either in the form of ssODN or circular double-stranded pDNA, is present at the site of the double-stranded break. Compared to the high (>50%) efficiencies achieved with NHEJ, HDR efficiencies are extremely low, typically around 20-30%, that too only when fused RNP-donor constructs are delivered in tandem via nucleofection. Several strategies have been demonstrated to overcome barriers to HDR: small molecule drugs that either block the NHEJ pathway or promote the HDR pathway, optimization of the length and sequence of the homology arms, modifying the protein structure of spCas9, and engineered fused donor template-RNP constructs. Whether DNA repair proceeds through error-prone repair or precise donor integration, is influenced by several variables: the cell cycle phase (S and G2 phases are favored), the delivery timing of the payloads and importantly, the composition and concentration of the repair templates relative to the guide RNA-Cas9 complex. Seeking to remain within constraints imposed by therapeutic applications, many researchers intentionally avoid pharmaceutical and biological manipulation during in vitro transfection, only for HDR efficiencies to fall below 1%.

As noted above, rational design of polyplexes for faithful DNA repair via HDR pathways requires optimization of three variables: the total nucleic acid dose, the proportion of sgRNA relative to the donor pDNA, and the polymer loading or N/P ratio. Factorial experiment design was used to simultaneously examine the effects of 1) the total nucleic acid dose, which was studied at 1.5 and 2 μg levels, 2) payload composition or the weight ratio of sgRNA to pDNA (w/w ratios of 2:1, 1:1, 1:2, 1:3, 1:4 and 1:5) and 3) N/P ratio (1, 1.25, 1.5, 2). The payload composition was varied while keeping the total nucleic acid dose fixed at 1.5 or 2 μg per well for a 24-well plate. Taken together, 48 conditions were evaluated in this experimental matrix, which identified the optimal conditions for HDR editing (FIG. 13B).

The relative frequencies of NHEJ and HDR was quantified by measuring mCherry and GFP expression, respectively. From flow cytometric measurements, it was determined that both the rate of integration of the donor plasmid (quantified via GFP readouts) as well as the formation of random indels (measured via mCherry expression) were highest when the nucleic acid loading was maximum (2 μg/well for a 24-well plate). Additionally, a complex non-monotonic relationship was noted between HDR frequency and the payload composition, wherein both sgRNA-dominant payloads (2:1) and pDNA-dominant payloads (1:5) conditions resulted in extremely low HDR frequencies (<0.1%) while intermediate payload compositions (1:2 and 1:3 w/w mixtures) resulted in the highest GFP expression (0.7%). mCherry expression was also highest at intermediate compositions, suggesting that both RNP and pDNA incorporation within polyplexes is highest at this mixing ratio.

Payload optimization was further investigated by benchmarking the HDR performance of the hit polymer DIP50H50 against commercial controls at the optimized polyplex formation conditions of 2 μg nucleic acid dose composed of a 1:2 w/w ratio of sgRNA and donor pDNA. The mCherry and GFP expression were measured, indicative of NHEJ and HDR editing respectively, in cells treated with DIP50H50 at N/P ratios of 1.25, 1.5, 1.75 and 2. Lipofectamine 2000 and JetPEI were also included as positive controls (FIG. 13D). While the use of JetPEI resulted in almost no HDR-edited cells, Lipofectamine 2000 was the only reagent where more than 2% of the cell population was GFP-positive. GFP expression did not exceed 0.7% when DIP50HEMA50 polymers were used to deliver HDR constructs, consistent with the results observed during the payload optimization experiment.

While not wishing to be bound by any theory, the causes underlying low HDR frequencies appear to originate in cellular processes rather than polymeric design; transfection was not synchronized with cell cycle, nor were HDR-promoting drugs employed to bias editing in favor of gene insertion. Despite the absence of biological and chemical intervention, a substantial pool of cells was obtained that underwent the HDR editing pathway, a cell population that can subsequently be sorted and expanded thereby enriching the pool of precisely edited cells and fulfilling therapeutic demands.

The present disclosure illustrates a process in which a polymer can be rapidly discovered that co-delivers therapeutic biological cargoes with contrasting physical characteristics and biological functions, a capability that is highly critical for genome editing applications such as homology-directed repair. Screening a 43-polymer library for pDNA transfection, a lead structure was identified, DIP50H50, that could enhance transient transfection more efficiently than JetPEI.

The present disclosure is further directed to methods for delivering the biological agent bonded with the polymers described above to a cell or to a subject. For example, after a composition including the polymers and biological agent payload bonded thereto is applied to the cell, the polyplexes are delivered into the cell and the biological agent payload disassociates partially or completely from the polymeric carriers and a therapeutic amount of the biological agent takes effect therein.

In various embodiments, which are not intended to be limiting, the compositions may be administered to a cell in vitro by removing a cell from a subject, culturing the cells, applying to the cells a composition including polymer vehicles and bonded biological agent to deliver a therapeutic amount of the biological agent into at least a portion of the cells, and optionally re-introducing the cell to the subject.

In another embodiment, a tissue cell therapy technique may be used in which a tissue sample is removed from a subject, a composition including a polymer and an bonded biological agent is applied to the tissue to deliver a therapeutic amount of the biological agent to modify a selected cell or region of the tissue, and the modified tissue is transplanted into the subject.

In another embodiment, a composition including a polymer and an associated biological agent is administered to a subject in vivo via direct injection into the bloodstream such that a therapeutic amount of the biological agent is delivered into desired target cells of the subject. In various embodiments, for in vivo administration a delivery device can be used to facilitate the administration of any composition described herein to a subject, e.g., a syringe, a dry powder injector, a nasal spray, a nebulizer, or an implant such as a microchip, e.g., for sustained-release or controlled release of any formulation described herein.

The copolymers described herein are configured to bind with a biological agent. In various embodiments, which are not intended to be limiting, the biological agent is chosen from a peptide fragment, nuclease, a nucleic acid encoding a nuclease, oligo nucleotide, a protein, peptide, a DNA editing template, guide RNA, a therapeutic agent (such as, for example, a drug), a plasmid DNA encoding protein, siRNA, monoclonal antibodies, Cas9 mRNA, and mixtures and combinations thereof. In various embodiments, the polymers are configured to bind with plasmid DNA (pDNA), which encode protein (fluorescence or therapeutic); pDNA encode Cas9 nuclease and/or sgRNA; mRNA that encodes for proteins (fluorescence or therapeutic), Cas9 nuclease and a separate sgRNA, a ribonucleoprotein (RNP) that in some embodiments includes recombinant Cas9 protein precomplexed directly with a sgRNA, and mixtures and combinations thereof.

In various embodiments, peptide fragments include two or more amino acids covalently linked by at least one amide bond (i.e. a bond between an amino group of one amino acid and a carboxyl group of another amino acid selected from the amino acids of the peptide fragment). The terms polypeptide and peptide fragments are used interchangeably. The term peptide fragment includes salts thereof, including pharmaceutically acceptable salts. For example, in some embodiments the peptide fragments may include pDNA encoded fluorescence or therapeutic proteins.

In various embodiments, DNA editing templates include an exogenous strand of DNA that bears homology arms to a section of genomic DNA that has been cut by a nuclease (for example, CAS9, TALEN or zinc finger) along with an intervening sequence between these homology arms that differs with the natural segment of genomic DNA that has been cut. This intervening segment selves as the template for repair of the cut genomic DNA and, in so doing, the cell corrects its own DNA to match that of the DNA template. The DNA template may be included in a single DNA expression vector that also encodes the nuclease.

The term guide RNA includes an engineered, non-naturally occurring Clustered Regularly Interspaced Short Palindromic Repeats (CRISPR) guide RNA that hybridizes with a target nucleic acid sequence of interest.

The term Cas9 mRNA includes a nucleotide sequence encoding a Type-II Cas9 protein, pDNA that encodes Cas9 protein, and pDNA that encode sgRNA. The CRISPR-Cas system is useful for precise editing of genomic nucleic acids (e.g., for creating null mutations). In such embodiments, the CRISPR guide RNA and/or the Cas enzyme may be expressed. For example, a composition containing only the guide RNA can be administered to an animal or cells transgenic for the Cas9 enzyme. Similar strategies may be used (e.g., designer zinc finger, transcription activator-like effectors (TALEs) or homing meganucleases).

The CRISPR-Cas system is known in the art for deleting, modifying genome sequences or incorporating transgenes. Transgene refers to any nucleotide sequence, particularly a DNA sequence, that is integrated into one or more chromosomes of a host cell by human intervention, such as by the methods of the present invention. For example, a transgene can be an RNA coding region or a gene of interest, or a nucleotide sequence, preferably a DNA sequence, that is used to mark the chromosome where it has integrated or may indicate a position where nucleic acid editing, such as by the CRISPR-CAS system, may occur. In this situation, the transgene does not have to include a gene that encodes a protein that may be expressed.

A gene of interest is a nucleic acid sequence that encodes a protein or other molecule, such as a RNA or targeting nucleic acid sequence, that is desirable for integration in a host cell. The gene of interest may include a 1, 2, 3, 4, 5, 6, 7, 8, 9, 10, or more genes of interest.

Genes of interest are useful for modulating the expression and/or activity of target biomolecules, either within the transduced cell or expressed for secretion outside of the transduced cell. Generally, genes of interest may be nucleic acids themselves or encode a polypeptide, a naturally-occurring binding partner of a target of interest, an antibody against a target of interest, a combination of antibodies against a target of interest and antibodies against other immune-related targets, an agonist or antagonist of a target of interest, a peptidomimetic of a target of interest, a peptidomimetic of a target of interest, a small RNA directed against or a mimic of a target of interest, and the like. Such modulators are well known in the art and include, for example, an antisense nucleic acid molecule, RNAi molecule, shRNA, mature miRNA, pre-miRNA, pri-miRNA, miRNA, anti-miRNA, or a miRNA binding site, or a variant thereof, or other small RNA molecule such as a Piwi RNA, triplex oligonucleotide, ribozyme, coding sequence for a target of interest. Such agents modulate the expression and/or activity of target biomolecules, which includes any decrease in expression or activity of the target biomolecule of at least about 30% to about 99% or more as compared to the expression or activity of the target biomolecule which has not been targeted by a modulating agent.

In one embodiment, the gene of interest is useful for expressing and/or enhancing the activity of a nucleic acid or protein of interest. For example, the gene of interest may encode a protein or other molecule the expression of which is desired in the host cell. Such protein-encoding nucleic acid sequences are not particularly limited and are selected based on the desired exogenous perturbation desired. Thus, the gene of interest includes any gene that the skilled practitioner desires to have integrated and/or expressed. For example, exogenous expression of proteins related to autoimmune, allergic, vaccination, immunotolerance, cancer immunotherapy, immune exhaustion, immunological memory, or immunological epitope responses may be used. The gene of interest encode a protein or be a nucleic acid that serves as a marker to identify cells of interest or transduced cells. The gene of interest may encode a protein that modifies a physical characteristic of the transduced cell, such as a protein that modifies size, growth, or eventual tissue composition. In another example, the gene of interest may encode a protein of commercial value that may be harvested. Generally, the gene of interest is operatively linked to other sequences that are useful for obtaining the desired expression of the gene of interest, such as transcriptional regulator sequences like inducible promoters, as described further below.

In another embodiment, the gene of interest is useful for inhibiting the expression and/or activity of a nucleic acid or protein of interest. For example, target biomolecule expression and/or activity, such as an RNA coding region, may be reduced or inhibited using inhibitory RNAs. An RNA coding region is a nucleic acid that may serve as a template for the synthesis of an RNA molecule, such as an siRNA. RNA interference (RNAi) is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see, for example, Coburn and Cullen (2002) J. Virol. 76:9225), thereby inhibiting expression of the target biomarker nucleic acid. In one embodiment, the RNA coding region is a DNA sequence. The ability to down-regulate a target gene has many therapeutic and research applications, including identifying the biological functions of particular genes. Moreover, such inhibition may be achieved in screening assays that take advantage of pooling techniques, whereby groups of about 2 to about 100, or more, or any number or range in between, of RNA inhibitory agents are transduced into cells of interest. Suitable inhibitory RNAs include, but are not limited to siRNAs, shRNAs, miRNAs, Piwis, dicer-substrate 27-mer duplexes, single-stranded interfering RNA, and the like.

siRNAs typically refer to a double-stranded interfering RNA. In addition to siRNA molecules, other interfering RNA molecules and RNA-like molecules may be used. Examples of other interfering RNA molecules that may to inhibit target biomolecules include, but are not limited to, short hairpin RNAs (shRNAs), single-stranded siRNAs, microRNAs (miRNAs), piwiRNA, dicer-substrate 27-mer duplexes, and variants thereof containing one or more chemically modified nucleotides, one or more non-nucleotides, one or more deoxyribonucleotides, and/or one or more non-phosphodiester linkages. Typically, all RNA or RNA-like molecules that may interact with transcripts RISC complexes and participate in RISC-related changes in gene expression may be referred to as interfering RNAs or “interfering RNA molecules.

Suitable interfering RNAs may readily be produced based on the well-known nucleotide sequences of target biomolecules. In various embodiments interfering RNAs that inhibit target biomolecules may comprise partially purified RNA, substantially pure RNA, synthetic RNA, recombinant produced RNA, as well as altered RNA that differs from naturally-occurring RNA by the addition, deletion, substitution, and/or alteration of one or more nucleotides. Such alterations may include, for example, addition of non-nucleotide material, such as to the end(s) of the interfering RNAs or to one or more internal nucleotides of the interfering RNAs, including modifications that make the interfering RNAs resistant to nuclease digestion. Such alterations result in sequences that are generally at least about 80%, or more, or even 100% identical to the sequence of the target biomolecule. When the gene to be down regulated is in a family of highly conserved genes, the sequence of the duplex region may be chosen with the aid of sequence comparison to target only the desired gene. On the other hand, if there is sufficient identity among a family of homologous genes within an organism, a duplex region may be designed that would down regulate a plurality of genes simultaneously.

Ribonucleoproteins (RNP) can be assembled by complexing spCas9, or other purified CRISPR-associated nucleases along with single guide RNA strands (sgRNA) that can be generated via chemical synthesis, plasmid generation or in vitro transcription. In this disclosure, sgRNA has been chemically modified to improve stability and prevent intracellular degradation. RNPs are typically about 8 nm in radius and possess an electrostatic charge of around −17.5 mV, ensuring their electrostatic assembly with polymeric vehicles. Compared to plasmid-based genome editing methods, the use of RNPs minimizes off-target effects since the nucleases will not persist in the cell over long durations but will degrade eventually. In some cases, RNPs are more suitable modality for performing genome editing of cells that are more challenging to transfect, such as primary cells. Moreover, co-delivery of RNPs and donor DNA can facilitate precise gene repair through the insertion of desired DNA sequences at the site of the sequence-specific DNA break mediated by the RNP.

In another aspect, the present disclosure is directed to compositions including the copolymer polyplexes described above which have been dispersed in an aqueous solution. In some embodiments, the polyplexes may be added to a liquid carrier and stored in liquid form until needed, or alternatively may be dried and introduced into and dispersed in the liquid carrier prior to administration to a subject.

In some embodiments the liquid carrier is a pharmaceutically acceptable carrier, which refers to a pharmaceutically-acceptable material, composition or vehicle for administration of a biological agent described herein. Pharmaceutically acceptable carriers include any and all solvents, dispersion media, coatings, antibacterial and antifungal agents, isotonic and absorption delaying agents, and the like which are compatible with the activity of the biological agent and are physiologically acceptable to the subject.

Some non-limiting examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, methylcellulose, ethyl cellulose, microcrystalline cellulose and cellulose acetate; powdered tragacanth; malt; gelatin; lubricating agents, such as magnesium stearate, sodium lauryl sulfate and talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol (PEG); esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; bulking agents, such as polypeptides and amino acids serum component, such as serum albumin, HDL and LDL; C2-C12 alcohols, such as ethanol; and other non-toxic compatible substances employed in pharmaceutical formulations.

Wetting agents, coloring agents, release agents, coating agents, sweetening agents, flavoring agents, perfuming agents, preservative and antioxidants can also be present in the formulation.

Pharmaceutically acceptable carriers can vary in a formulation described herein, depending on the administration route. The formulations described herein can be delivered to a cell or an organism via any administration mode known to a skilled practitioner. For example, the formulations described herein can be delivered in a systemic manner, via administration routes such as, but not limited to, simply applying the composition to an exterior surface of a cell, oral, intravenous, intramuscular, intraperitoneal, intradermal, and subcutaneous. In some embodiments, the compositions described herein are in a form that is suitable for injection. In other embodiments, the formulations described herein are formulated for oral administration.

In some embodiments, the liquid carrier for the polyplexes can be a solvent or dispersing medium, containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutical carrier ca be a buffered solution (e.g., PBS).

The formulations can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations. With respect to formulations described herein, however, any vehicle, diluent, or additive used should be biocompatible with the biological agents described herein.

The copolymers and methods of the present disclosure will now be further described in the following non-limiting examples.

### EXAMPLES

### Example 1—Experimental Parameters for Polymer Synthesis

TABLE 2
Summary of reactant stoichiometry & solvent composition

| | | | Cat. | | | Total | | |
|---|---|---|---|---|---|---|---|---|
| | | | mono. | | | mono. conc. | | |
| Polymer | m | n | (% mol) | CTA | Mono:CTA:Ini | [M] | Solvent | dn/dc |
| p(DEAEMAₘ-st-MPCₙ) | 98 | 0 | 100 | CPAD | 70:1:0.1 | 0.9 | DMF | 0.1328 |
| | 53 | 32 | 75 | CPAD | 70:1:0.1 | 0.9 | 1:1 v/v | 0.1069 |
| | 37 | 58 | 50 | CPAD | 70:1:0.1 | 0.9 | EtOH & | 0.1023 |
| | 22 | 91 | 25 | CPAD | 70:1:0.1 | 0.9 | H₂O | 0.0997 |
| p(AEMAₘ-st-MPCₙ) | 67 | 0 | 100 | CPP | 70:1:0.1 | 0.5 | 1:1 v/v | 0.1855 |
| | 50 | 23 | 75 | CPP | 70:1:0.1 | 0.5 | CH₃COO⁻Na⁺ | 0.141 |
| | 43 | 51 | 50 | CPP | 100:1:0.1 | 0.25 | &EtOH | 0.1437 |
| | 19 | 61 | 25 | CPP | 70:1:0.1 | 0.5 | | 0.141 |
| p(DIPAEMAₘ-st-MPCₙ) | 80 | 0 | 100 | CPAD | 70:1:0.1 | 0.9 | DMF | 0.1528 |
| | 81 | 33 | 75 | CPAD | 70:1:0.1 | 0.9 | 1:1 v/v | 0.1274 |
| | 65 | 64 | 50 | CPAD | 70:1:0.1 | 0.9 | EtOH & | 0.1193 |
| | 22 | 91 | 25 | CPAD | 70:1:0.1 | 0.9 | H₂O | 0.1032 |
| p(DMAEMAₘ-st-MPCₙ) | 110 | 0 | 100 | CPP | 70:1:0.1 | 1 | DMSO | 0.174 |
| | 25 | 9 | 90 | CPP | 100:1:0.1 | 0.25 | 1:1 v/v | 0.1277 |
| | 48 | 32 | 65 | CPAD | 70:1:0.1 | 1 | EtOH & | 0.1252 |
| | 17 | 47 | 40 | CPAD | 70:1:0.2 | 1 | H₂O | 0.1473 |
| | 0 | 24 | 0 | CPAD | 70:1:0.1 | 0.9 | | 0.0885 |
| p(DEAEMAₘ-st-PEGₙ) | 74 | 20 | 75 | CEP | 70:1:0.1 | 1.8 | DMSO | 0.1270 |
| | 53 | 42 | 50 | CEP | 70:1:0.1 | 1.8 | | 0.1237 |
| | 36 | 65 | 25 | CEP | 70:1:0.1 | 1.8 | | 0.1220 |
| p(AEMAₘ-st-PEGₙ) | 85 | 35 | 75 | CEP | 100:1:0.25 | 0.5 | 2:1 v/v | 0.1369 |
| | 90 | 120 | 25 | CEP | 100:1:0.25 | 0.5 | CH₃COO⁻Na⁺ | 0.1246 |
| | 44 | 79 | 50 | CEP | 70:1:0.4 | 3.5 | &Dioxane | 0.1311 |
| p(DIPAEMAₘ-st-PEGₙ) | 36 | 20 | 75 | CEP | 70:1:0.1 | 1.8 | DMSO | 0.1342 |
| | 29 | 33 | 25 | CEP | 70:1:0.1 | 1.8 | | 0.1289 |
| | 27 | 82 | 50 | CEP | 70:1:0.1 | 1.8 | | 0.1240 |
| p(DMAEMAₘ-st-PEGₙ) | 26 | 9 | 75 | CEP | 70:1:0.1 | 1 | DMSO | 0.185 |
| | 14 | 12 | 50 | CEP | 70:1:0.1 | 1 | | 0.185 |
| | 9 | 67 | 25 | CEP | 70:1:0.1 | 1 | | 0.0995 |
| | 0 | 7 | 0 | CEP | 70:1:0.1 | 1.8 | | 0.12 |
| p(DEAEMAₘ-st-HEMAₙ) | 66 | 21 | 75 | CEP | 70:1:0.1 | 1.8 | DMSO | 0.1322 |
| | 47 | 47 | 25 | CEP | 70:1:0.1 | 1.8 | | 0.1317 |
| | 22 | 50 | 50 | CEP | 70:1:0.1 | 1.8 | | 0.1311 |
| p(AEMAₘ-st-HEMAₙ) | 85 | 54 | 75 | CEP | 100:1:0.25 | 1 | 1:1 v/v | 0.1616 |
| | 72 | 92 | 60 | CEP | 100:1:0.25 | 1 | CH₃COO⁻Na⁺ | 0.1485 |
| | 40 | 132 | 35 | CEP | 100:1:0.25 | 1 | &EtOH | 0.1487 |
| p(DIPAEMAₘ-st-HEMAₙ) | 61 | 33 | 75 | CEP | 70:1:0.1 | 1.8 | DMSO | 0.1472 |
| | 52 | 50 | 25 | CEP | 70:1:0.1 | 1.8 | | 0.1444 |
| | 27 | 80 | 50 | CEP | 70:1:0.1 | 1.8 | | 0.1382 |
| p(DMAEMAₘ-st-HEMAₙ) | 34 | 22 | 75 | CEP | 70:1:0.1 | 1 | DMSO | 0.195 |
| | 32 | 45 | 65 | CEP | 70:1:0.1 | 1 | | 0.1432 |
| | 65 | 145 | 25 | CEP | 70:1:0.1 | 1 | | 0.0971 |
| | 0 | 60 | 0 | CEP | 70:1:0.1 | 1.8 | | 0.13 |

CTA: chain transfer agent
Cat. mono. (mol %): mole % of cationic monomer used in monomer feed.
Mono:Ini:CTA: Mole ratio of monomer to CTA to polymerization initiator (V-501)
CPAD: 4-cyano-4-(phenylcarbonothioylthio)pentanoic acid
CEP: 4-cyano-4-(ethylsulfanylthiocarbonyl)sulfanyl pentanoic acid
CPP: 4-cyano-4-(propylsulfanylthiocarbonyl)sulfanyl pentanoic acid
CH3COO−Na+: Sodium acetate buffer solution, 3M.
EtOH: Ethanol
DMF: N,N dimethylformamide
DMSO: Dimethylsulfoxide

### Polymer Synthesis and Purification Procedures

All monomers, one of the chain transfer agents, 4-cyano-4-(phenylcarbonothioylthio)pentanoic acid (CPAD), and the V-501 polymerization initiator, 4,4-azobis(4-cyanovaleric acid), were purchased from Sigma Aldrich. The other chain transfer agent, 4-cyano-4-[(ethylsulfanylthiocarbonyl) sulfanyl] pentanoic acid (CEP), was purchased from Asta Tech (China) and used without further purification.

Liquid monomers were passed through a bed of basic alumina to remove polymerization inhibitors. The Carousel 12 parallel synthesizer (Radleys, UK) performed RAFT polymerization in high throughput, enabling the synthesis of the entire library. The quantities of CTA, initiator, monomers and the solvents dispensed are specified in Table 2 below.

In a typical polymerization run, the desired molar equivalents of monomers, solvents, CTA and initiators were dispensed into individual reaction modules of the Carousel 12 set up. All 12 reaction modules were simultaneously degassed using 3-4 freeze-pump-thaw cycles that till a vacuum level of 20-30 mTorr was achieved. After the final thaw, the reactor was heated to a temperature of 78° C. to initiate polymerization and then maintained under an inert nitrogen environment overnight. The next morning, the reaction mixtures were quenched and then slowly exposed to ambient atmosphere. Thereafter, 1-2 mL of 1N HCl was added to protonate cationic repeat units and the reaction mixtures transferred to dialysis bags (Spectrum Chemicals, NJ) with a molecular weight cut off of 3000 Da. The unreacted monomers and CTA were separated from the polymer using dialysis over 3-4 days in milliQ water with the dialysis medium being replaced twice daily. Finally, lyophilization (SP Scientific, PA) was performed for over two days to remove bound water and yield pure polymers.

### Polymer Characterization Procedures

### Nuclear Magnetic Resonance

Subsequent to purification, polymers were dissolved in D20 and NMR was performed on the Bruker Avance III HD 500 instrument. A total of 32 scans were acquired using a relaxation time of 10 seconds. Copolymer composition was evaluated by integrating characteristic NMR peaks from each monomer.

### Size Exclusion Chromatography

Size exclusion chromatography (Agilent, CA) was performed using refractive index and multiple angle light scattering detectors (Wyatt, Santa Barbara, Calif.) to determine the complete molecular weight distribution for all copolymers. The dn/dc values for homopolymers were determined using refractometry and the dn/dc of statistical copolymers was computed assuming additivity of refractive index increments, that is by using a weighted average approximation.

### pKa Measurements

An equivalence point seeking automated titrator, Orion star T901 pH titrator (Thermo Fisher) was employed for all titrations. Briefly, the polymer sample of interest was dissolved in milliQ water at a concentration of 0.5 mg/mL and the pH adjusted to 2 by the addition of HCl. A solution of 0.1 M NaOH was gradually dispensed in increments of 10-500 μL as determined by an equivalence point-seeking algorithm, until two equivalence/inflection points were observed. The data was exported and pKa were determined according to known procedures.

### ζ-Potential Measurements

The Malvern Zetasizer (Malvern Instruments, MA) was used to evaluate the ζ-potential of all polymers in the library through electrophoresis. Measurements were performed at a concentration of 1-2 mg/mL in PBS buffer or in water using a folded capillary measurement cell. Three to five measurements were acquired, and the average value reported in Table 1 above.

For electrokinetic characterization of the polyplexes, similar procedures were used. Polyplexes were formulated between pDNA and the hit polymer at N/P ratios of 1, 2.5, 5 and 10 and analyzed directly. For RNP payload, N/P ratios of 0.5, 1, 1.5 and 2 were studied. At least 3-5 measurements were collected per sample.

### Physical Characterization of Polyplexes

Polyplex Formulation: pDNA Payloads

Polymers were dissolved in ultrapure water to achieve the desired N/P ratios of 5, 10 and 20. Polyplexes were formed using an electronic multi-channel pipette (ClipTip 300) by controlled addition of polymer solution to an equal volume pDNA solution in sterile water. A pDNA concentration of 0.02 μg/μL and 0.05 μg/μL was employed for DLS and gel migration, respectively. The polyplexes were then incubated for 45 min at room temperature before further analysis. DLS measurements were performed in PBS whereas gel migration, zeta potential analysis and transfection formulations of pDNA polyplexes were completed in water.

### Polyplex Formulation: RNP Payloads

Synthetic single guide RNA (100 bp) was synthesized with a sequence of GCACCUAUAGAUUACUAUCCGUUUUAGAGCUAGAAAUAGCAAGUUAAAAU AAGGCUAGUCCGUUAUCAACUUGAAAAAGUGGCACCGAGUCGGUGCUUUU (Synthego, CA). spCas9 protein was ordered from Aldveron, ND. Polymer stock solutions were prepared in PBS at a concentration of 1 mg/mL and further diluted in PBS to achieve the desired N/P ratios (0.5,1,1.5, 2). Ribonucleoprotein complexes were assembled by adding sgRNA (0.039 mg/mL and 0.05 mg/mL for DLS and gel migration respectively) to equal volumes of Cas9 protein (0.19 mg/mL and 0.25 mg/mL for DLS and gel migration respectively), obtaining a 1:1 molar mixture of the Cas9 and sgRNA. RNP complexes were annealed for 10-15 min at room temperature before the addition of polymer. Thereafter, equal volumes of polymer solutions were added slowly and maintained at ambient temperature for 45 minutes.

### Gel Migration Assay

Gel casting was done using 0.6% agarose and 1.5% w/v agarose solutions in TAE buffer for pDNA and RNP payloads, respectively. To visualize nucleic acid bands, ethidium bromide was used at a concentration of 0.017% v/v to visualize pDNA migration towards the positive electrode. Around 20 μL polyplexes were loaded into each well after the addition of loading dye and gel electrophoresis was performed at 80 V over 60 minutes and imaged using a transilluminator (Fotodyne, IL) under UV light.

### Dynamic Light Scattering

Polyplexes were prepared at N/P ratios of 5, 10, and 20 for pDNA polyplexes and at N/P ratios of 1 and 2 for RNPs. Both formulations were prepared in D-PBS buffer (10 mM, pH 7.4, 137 mM NaCl) using multi-channel electronic pipettes. They were incubated at 23° C. for 45 minutes prior to acquisition of measurements. A DynaPro plate reader III (Wyatt Instruments, CA) was used to collect 5 acquisitions per sample. Auto-correlation functions with noisy baselines were filtered out using an automated baseline-filtering process and the polyplex size distributions were computed using regularization models. Intensity weighted average hydrodynamic radii have been reported after being averaged over 3-5 measurements.

### Biological Assays

### Cell Culture

The HEK293 cell line engineered with traffic light reporter system was used to assess both RNP and pDNA delivery by all polymers in our combinatorial library. To obtain a stable cell line, 24 single cell clones were prepared at Genome Engineering Shared Resource (Minneapolis, Minn.) and screened for mCherry expression upon Lipofectamine-meditated RNP delivery. The selected sub-clone was used for all gene editing studies. Cells were seeded at 50,000 cells/well in DMEM supplemented with 10% FBS in 48-well plates (Corning, MA). Cells were cultured for 24 hours at 37° C. and 5% CO₂ to allow the cells to adhere to the plate and achieve confluencies between 30-50%. For both RNP and pDNA delivery experiments, the total volume of the polyplex solution added was 150 L (30 L of polyplex solution and 120 μL of Opti-MEM). After 4 hours, a further 0.5 mL of FBS-supplemented DMEM was added to each well. Twenty-four hours after transfection, old media was aspirated and replaced with fresh DMEM. Forty-eight hours later, the cells were analyzed either using plate reader (for plasmid transfection) or using wide field microscopy (for RNP transfection) or using flow cytometry (for both payloads). Manufacturer's protocols were executed for all commercial controls.

### Plate Reader

48 hours after transfection with pDNA-polymer complexes, cells were trypsinized and resuspended in optically clear DMEM formulated without FBS and phenol red. This suspension was transferred to a 96-well opaque plate for plate reader measurements. The Synergy H1 plate reader (Biotek, CA) was used to measure GFP intensity in each well using an area scan. Measurements were performed in triplicate and the background readings from untreated cells were subtracted from each measurement.

Transfection Procedures for RNP and pDNA Payloads

For RNP transfection, all polymer stock solutions were formulated at a concentration of 1 mg/mL spCas9 (Aldevron, ND,) and sgRNA (Synthego, CA) solutions were prepared at concentrations of 0.039 mg/mL and 0.19 mg/mL respectively and the ribonucleoprotein formed by slow addition of sgRNA to spCas9 and annealing for 15 minutes. Thereafter, polymers were diluted and slowly added to the RNP to achieve N/P ratios of 1 and 2. Polyplexes were diluted in OptiMEM before introducing them to the cells. For a 24-well plate, a nucleic acid loading of 1 μg/mL sgRNA was employed. Two days after transfection, media was exchanged with Fluorbrite (Thermo Fisher) supplemented with 2.5% v/v FBS. Cells were stained by adding 1 drop Nucelobrite (Thermo Fisher) to each well to visualize nuclear outlines and facilitate cell counting. Live cells were imaged using a Zeiss confocal microscope (Zeiss, Switzerland) equipped with a motorized stage and automated image acquisition features. 4-20 fields of view were acquired for each sample group using uniform exposure time and gain settings in the mCherry and Hoechst channels. The images were analyzed using Cell profiler using previously detailed procedures to quantify mCherry expression.

For pDNA transfection, cells were seeded in 24-well plates at 50,000 cells/well a day prior to transfection in 1 mL DMEM/well containing 10% HI FBS. To formulate polyplexes, 175 μL polymer solution was added 175 μL pZSGreen-N1 (Aldevron, ND) solution (20 ng/μL) and incubated at room temperature for 45 minutes. OptiMEM (700 L) was added to each polyplex sample and 300 L polyplex solution was then added to each well after aspirating old media. After 4 h, 1 mL DMEM containing 10% HI FBS was added to each well. Cell culture media was replaced 24 h after transfection and analysis (flow cytometry or plate reader measurements) completed after 2 days.

### Flow Cytometry

The transfection procedures for pDNA and RNP described earlier were again followed here, with the only change being that 24-well plates were used for validation studies instead of 48-well plates, and the quantities of payloads and polymers doubled accordingly. Two days after transfection, cells were trypsinized and the cell suspension centrifuged at 1100 RPM and 4° C. for 10 mins. The supernatant was completely removed, and the cell pellet was resuspended in a 200 μL solution of PBS+2% FBS+400 nM Calcein Violet (Thermo Fisher). Cells were incubated in ice for 30 minutes and vortexed prior to flow cytometry. For measuring GFP expression in transiently transfected HEK cells, the 405 nm and 488 nm laser lines were used on the ZE 5 flow cytometer (Biorad Inc, CA). For evaluating mCherry production in edited cells (after RNP delivery), the 405 nm and 560 nm laser lines were used. Single live cells were used for analysis and gating schemes are furnished in the SI. At least 80,000 events were collected per sample for RNP delivery and HDR studies.

### Toxicity Studies

Transfection was performed in 48-well plates according to procedures for RNP and pDNA delivery described previously. Two days after transfection, cell culture media was replaced with a 2% solution of CCK-8 (Dojindo) in Fluorbrite. Thereafter, cells were incubated for 4 hours at 37° C. and 5% CO₂ and absorbance measured at 450 nm at a gain of 90 using the Synergy H1 plate reader (Biotek, CA). Measurements of the CCK-8 solution without cells were collected and this blank reading was subtracted from all data points. Absorbance values were normalized to untreated cells. Six wells were employed per condition.

### DNA Sequencing

The DNA of transfected cells was extracted using the manufacturer's protocol of Quick-Extract DNA extraction solution (Lucigen, WI). Thereafter, the extracted templates were analyzed using Nanodrop spectrophotometer (Thermo Fisher) to verify DNA quality before being PCR-amplified using the AccuPrime Taq DNA Polymerase kit (Thermo Fisher).

Primer sequences used were: 5′ AGACCACCCCCATGTACAAA 3′ and 5′ GGAAAACCCTTCCTGGTTTC 3′. Primers were ordered from Integrated DNA Technologies (IDT, Skokie, Ill.) and dissolved in ultrapure water. PCR products were purified with 1 wt % agarose gel electrophoresis and excised gel fragments purified using Monarch DNA gel extraction kit (New England BioLabs, MA). Purified DNA products were eluted in ultra-pure water and analyzed using Sanger sequencing after the addition of primer. Sequencing results were analyzed using TIDE assay and Synthego's ICE program.

### HDR Experiments

For homology-directed repair, HEK293 cells were co-transfected with a mixture of RNP and donor plasmid payloads. the total mass of the sgRNA and the DNA repair template was kept fixed at either 1.5 μg per well or 2 μg for a 24-well plate. However, their weight ratio was varied systematically from 2:1 to 1:5 in order to identify the formulation conditions that would maximize the frequency of HDR events. The evidence indicated that 2 μg loading per well and 1:2 w/w ratio of sgRNA:pDNA were the optimal conditions using these flow cytometric measurements.

In a typical experiment, RNP complexes would be annealed by adding sgRNA solution to spCas9 solution in equal volumes, as described previously. Within 15 minutes of RNP formation, an equal volume of the donor plasmid solution would be added and allowed to equilibrate for 5 minutes. The polymer solution (diluted to the desired N/P ratio in D-PBS) would be slowly introduced into an equal volume of the payload mixture and incubated for 45 minutes at ambient temperature. Finally, this mixture would be diluted in twice the volume of OptiMEM and added slowly to cells. Cells would be plated 24 hours prior to transfection at a density of 50,00 cells/mL. DMEM supplemented with FBS would be added 4 hours after transfection and replaced 24 hours after transfection. Cells would be passaged while approaching 80% confluency (roughly every 2 days) before being analyzed using flow cytometry on the seventh day after transfection.

### Example 2—Data Science Analysis of Copolymers

Nine polymer descriptors were included in the analysis, of which 7 were derived through routine experimental methods: polymer composition (% incorporation of the cationic monomer), the degree of polymerization (Mₙ), the N/P ratio of polyplex formulations, the pKa and ζ-potential values of the polymers, the polyplex diameter as well as the binding state of the RNP polyplexes observed during gel migration assays.

Additionally, log P values were calculated for each of the 43 polymers from molecular simulations of oligomeric models. While log P values roughly approximate experimentally derived partition coefficients, in some cases the underlying trends in polymer hydrophobicity will be sufficiently captured through the computational calculation.

Finally, the cooperativity during polymer deprotonation was quantified by computing the Hill coefficient (n_{Hill}) from pKa titration curves. It is known that for polymers with high n_{Hill}, a strong driving force exists for hydrophobic collapse, which causes the deprotonation of a single amine moiety to trigger spontaneous deprotonation of neighboring amine groups, ensuring extremely high sensitivity to small increases in pH and rapid hydrophobic collapse. While the pKa is an equilibrium constant that measures the cationic polymer's preference for existing in a highly protonated state, the n_{Hill} on the other hand, is a pKa-independent parameter quantifying the polymer's pH-responsiveness, or how easily it switches from 100% protonation to complete deprotonation.

The resulting dataset is high-dimensional and complex, since the above 9 polymer descriptors are not perfectly independent, but intertwined in complex ways. For instance, n_{Hill} and c log P are both highly correlated since lengthening the alkyl chains substituents within the tertiary amine groups of cationic monomers, is likely to cause both of these parameters to increase. Similarly, the RNP binding parameter is highly dependent on both the pKa and the polymer ζ-potential, given that the degree of amine protonation influences both electrophoretic mobility as well as Coulombic interactions with the RNP payload. Recognizing the entangled nature of our polymer descriptors as well as the limitations of employing a narrow univariate lens for data exploration, it was assumed that sophisticated analytical approaches such as PCA would reveal the contribution of each of the above 9 descriptors and also examine whether synergistic combinations of polymer attributes shape transfection.

To deal with the dual challenges posed by high dimensionality as well as the complex correlations between descriptors, PCA was performed to simplify data visualization. PCA reprocesses 9 descriptors into new variables termed principal components (PCs), which are linear combinations of the original set of polymer descriptors.

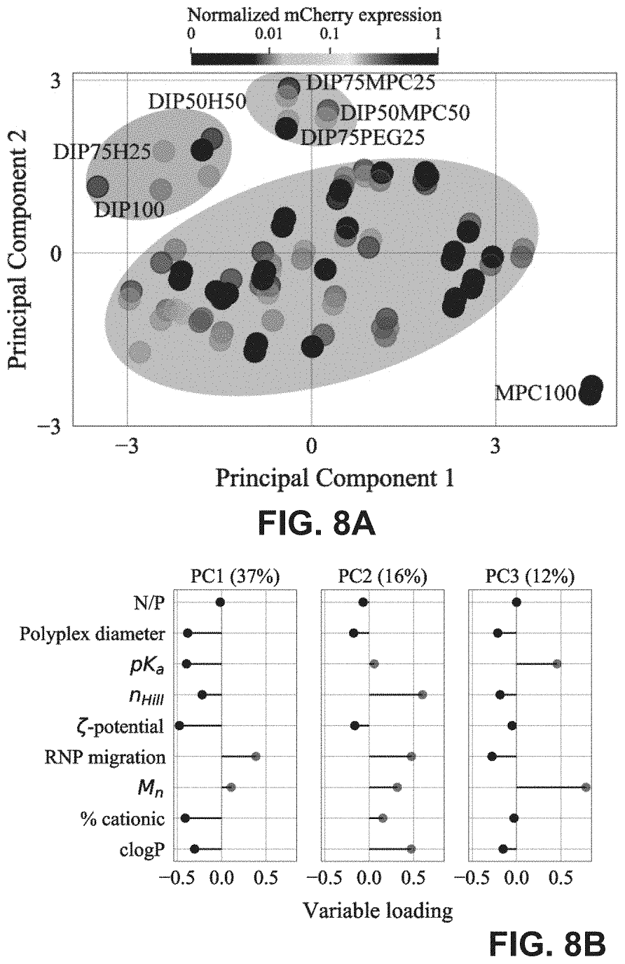

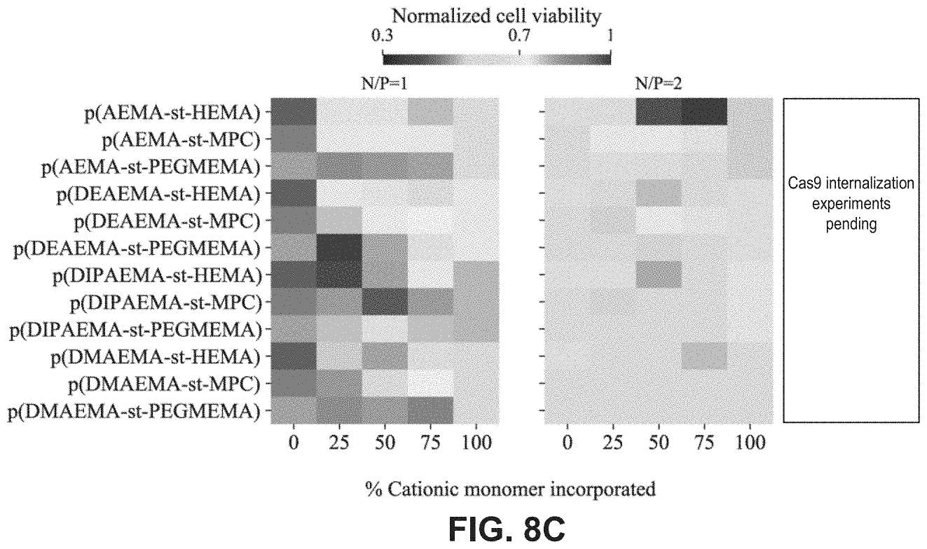

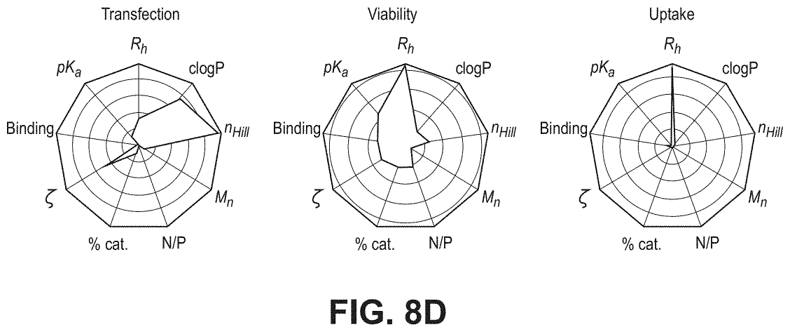

The composition of the first three PCs is represented in FIG. 8B where the contribution of each of the 9 descriptors is compared to the respective PCs. Unlike the unprocessed descriptors, PCs are perfectly orthogonal to each other and also represent coordinates along which the variation in mCherry expression can be optimally represented. Through PCA, this 9-dimensional space of correlated descriptors was compressed into a 3-dimensional space without losing any structure-function correlations latent within the unprocessed data. The first two PCs for the mCherry dataset are shown in FIG. 8A, where it is shown that the greatest variation in mCherry expression occurs along PC1, with increasing mCherry expression occurring with decreasing PC1.

Instead of visualizing mCherry expression as a complex function of 9 variables, differences and similarities can be determined between polymers in the library by plotting data in 3-dimensional PC space instead of 9-dimensional descriptor space. The copolymer DIP50H50 occupies a cluster in PC space that is diagonally opposite that of the hydrophilic homopolymers MPC100, H100 and PEG100, all of which were ineffective in mediating intracellular RNP delivery and NHEJ editing. In contrast, the close proximity of DIP50H50 with structurally similar analogs such as DIP100, DIP75H25, DIP50MPC50, DIP75PEG25 and DIP75MPC25 is particularity interesting since it suggests that their physicochemical properties overlap considerably with that of DIP50H50, despite significant differences in chemical composition.

Among this family of related polymers, near-identical polyplex size distributions were observed, RNP binding behavior and Hill coefficients, indicating that a very fine line separates the hit polymer from marginal performers such as DIP100. Despite the wide gap in mCherry expression levels between DIP50H50 and the rest of the library, five of these “near-miss” polymers happen to possess to a limited degree, the mix of unique physicochemical features that made DIP50H50 such an effective vehicle for RNP delivery.

Next, the scope of the statistical modeling efforts was expanded to include additional biological responses such as toxicity and cellular internalization of payloads, instead of restricting the analysis to editing efficiency, i.e., mCherry expression alone. To compare toxicity associated with each polyplex formulation, CCK-8 measurements were completed, normalized readings to unperturbed cells and plotted responses in the form of a heat map (FIG. 8C). For payload uptake, labeled RNPs were used that were constituted from GFP-fused spCas9 constructs, and formulated polyplexes using labeled RNPs and 43 polymers in the library at 2 N/P ratios. RNP transfection was performed using procedures similar to those used for unlabeled RNPs.

24 hours post-transfection, extensive washing with CellScrub was performed to remove extracellularly bound polyplexes and GFP intensity levels were measured for all 86 formulations using flow cytometry (FIG. 8C). Although DIP50H50 was not alone in causing severe toxicity or effecting high cellular internalization of RNP payloads, it was the sole polymer to achieve efficient NHEJ editing, suggesting that the structural determinants for transfection, toxicity and internalization were not identical.

To further probe this possibility, three random forest ensemble (RFE) models were developed to implement the following classification tasks: is each polymer a hit/not a hit (hit criterion: normalized mCherry >0.4), toxic/not toxic (criterion for being labeled toxic: bottom 5 percentile), and responsible for high/low internalization (criterion: <5% uptake). RFE is a classification algorithm that constructs ensembles of “decision trees,” wherein each decision tree examines distinct cross sections of the data as well as a different selection of polymer descriptors to classify each polymer according to the criteria specified above. This randomization process ensures that the classification rules developed by each decision tree is diverse, that multiple explanations are considered and that a broad representative picture is developed to describe trends in the data. By aggregating how heavily each decision tree in each forest relies on a given polymer descriptor to classify polymers correctly, the feature importance (scaled to 1) of each of the descriptors can be compared across the three datasets (transfection, toxicity and uptake).

Visualizing relative feature importance in the form of a radar plot (FIG. 8D), strikingly distinct trends were observed across transfection, toxicity and uptake. Hydrophobicity-linked parameters such as c log P and n_{Hill} were identified as the primary structural drivers of high editing efficiency while bulkier polyplexes and polymers with high charge densities (ζ-potential) also contributed marginally. While engineering polymers with high hydrophobicity has frequently been touted as a polymer design strategy essential to achieve efficient delivery, no study has previously examined the role of cooperativity-enhanced deprotonation in enhancing nucleic acid delivery. While not wishing to be bound by any theory, presently available evidence indicates that polymers with high n_{Hill} may undergo rapid zipper-like deprotonation in the cytosol, resulting in instant payload unpackaging transfected cells. However, when contributors for toxicity and cellular internalization were considered, hydrophobicity-linked descriptors such as c log P and n_{Hill} had negligible impact.

Instead, the evidence indicated that polyplex diameter was the single most important descriptor, closely followed by electrostatics-linked descriptors such as RNP binding affinity, pKa and polymer ζ-potential. Although maximizing polyplex diameters and optimizing protonation degrees and electrostatic interactions may promote high cell uptake and cause severe toxicity, such polymers (e.g., AEMA75H25, AEMA50H50) may yet turn out to be ineffectual in releasing payloads within the cytosol if they do not satisfy the design constraints for c log P and n_{Hill}. To mediate efficient intracellular unpackaging, the c log P and the Hill coefficient should be maximized, by incorporating co-monomers of moderate hydrophilicity and tertiary amines with bulky hydrophobic substituents, while avoiding extremely hydrophilic monomers such as PEG and zwitterionic methacrylates such as MPC.

The statistical models this revealed that a single polymer descriptor cannot be used in isolation to guide the design of future libraries; rather, complex non-linear relationships between several molecular attributes shaped the structure-function landscape.

Further, PCA revealed that the properties responsible for DIP50H50's delivery efficacy were not unique to the hit polymer, but were also detected to some extent in structurally related analogs such as DIP100, indicating that the gap between the hit structure and the rest of the library was not as wide as initially suspected.

Finally, the physicochemical drivers were identified for transfection, toxicity and internalization using RFEs and concluded that while polymers resulting in high polyplex diameters and optimal pKa and ζ-potential values may mediate cellular internalization and even cause toxicity, successful payload unpacking can only be achieved by focusing on polymer hydrophobicity and engineering cooperative polymer deprotonation.

### Example 3

### Polyplex Formulation

The 43 polymers synthesized in Example 1 and characterized in Example 2 were used without further modification or purification. Polymer stock solutions were prepared by dissolution in ultrapure water and were sterilized via filtration. Stock solutions were further diluted to achieve concentrations leading to N/P ratios of 5, 10 and 20. Polyplexes were formed using an electronic multi-channel pipette (ClipTip 300, Thermo Fisher) by controlled addition of polymer solutions to equal volumes of pDNA solution in sterile water. The polyplexes were then incubated for 45 minutes at room temperature before further analysis. DLS measurements were performed in PBS whereas gel migration, ζ-potential analysis and polyplex preparation for transfection experiments were completed in water. The final pDNA concentration was 0.02 μg/μL and 0.05 μg/μL in samples employed for DLS and gel migration, respectively.

### Gel Migration Assays

Gel casting was performed using 0.6% agarose in 1×TAE buffer and ethidium bromide was used at a concentration of 0.017% v/v to visualize pDNA migration towards the positive electrode. Around 20 μL polyplexes were loaded into each well after the addition of loading dye and gel electrophoresis was performed at 80 V over 60 minutes and imaged using a transilluminator (Fotodyne, IL) under UV light. Gel migration assays were performed for all 43 polymers, all of which were complexed with pDNA payloads at N/P ratios of 5, 10, and 20, giving rise to 129 formulations.

### ζ-Potential Measurements

The Malvern Zetasizer (Malvern Instruments, MA) was used to evaluate the ζ-potential of polyplexes formulated using DIP50H50 and pZsgreen plasmid DNA payloads. Electrophoretic measurements were performed in water using a folded capillary measurement cell. Three to five measurements were acquired, and the average value reported. Polyplexes were formulated between pDNA and the hit polymer at N/P ratios of 1, 2.5, 5, and 10 and analyzed directly. Samples were diluted to achieve a final pDNA concentration of 50 ng/μL.

### Dynamic Light Scattering

Polyplexes were prepared at N/P ratios of 5, 10, and 20 in D-PBS (10 mM, pH 7.4, 137 mM NaCl) using multi-channel electronic pipettes as described in the previous section. They were incubated at 23 C for 45 minutes prior to acquisition of measurements. DynaPro plate reader III (Wyatt Instruments, CA) was used to collect 5 acquisitions per sample. Auto-correlation functions with noisy baselines were filtered out using an automated baseline-filtering process and the polyplex size distributions were computed using regularization models. Intensity weighted average hydrodynamic radii have been reported after being averaged over 3-5 measurements.

### Cell Culture

Consistent with Examples, 1-2, the HEK293 cell line was engineered with a traffic light reporter system to assess pDNA delivery by all polymers in our combinatorial library. Cells were seeded at 50,000 cells/well in DMEM supplemented with 10% HIFBS in 48-well plates (Corning, MA). Cells were cultured for 24 hours at 37 C and 5% CO₂ and passaged routinely around 80% confluency, roughly at a frequency of 3-4 days. Mycoplasma testing was completed every 3 months to ensure that cultures were free of contamination.

### Transfection Procedure

Cells were seeded in 24-well plates at 50,000 cells/well a day prior to transfection in 1 mL DMEM/well containing 10% HI-FBS. Polymer solutions were prepared to obtain concentrations appropriate to desired N/P ratios. To formulate polyplexes, 175 μL polymer solution was added 175 μL pZSGreen-N1 (Aldevron, ND) solution (20 ng/μL) and incubated at room temperature for 45 minutes. At the end of the incubation period, polyplexes were re-suspended in OptiMEM. About 700 L OptiMEM was added to each polyplex sample and 300 L polyplex solution was then added to each well after aspirating cell culture media.

Four hours after the addition of polyplexes suspended in OptiMEM, each well was supplemented with 1 mL DMEM containing 10% HI FBS. Cell culture media (DMEM+10% HI FBS) was replaced 24 h after transfection and analysis (flow cytometry or plate reader measurements) completed after 2 days. For JetPEI and Lipofectamine 2000, manufacturer protocols were implemented.

### Plate Reader Measurements of GFP Fluorescence Intensity

The transfection procedures outlined above were followed, with the only change being that 48-well plates were used instead of 24-well plates, and the quantities of payloads and polymers halved accordingly. 48 hours after transfection, cells were trypsinized and re-suspended in optically clear DMEM formulated without FBS and phenol red. This suspension was transferred to a 96-well opaque plate for plate reader measurements. The Synergy H1 plate reader (Biotek, CA) was used to measure GFP intensity in each well using an area scan. Measurements were performed in triplicate and the background readings from untreated cells were subtracted from each measurement.

### Flow Cytometric Measurements of GFP Expression

Two days after transfection, cells were trypsinized and the cell suspension centrifuged at 1100 RPM and 4 C for 10 mins. The supernatant was completely removed, and the cell pellet was resuspended in a 200 uL solution of PBS+2% FBS+400 nM Calcein Violet (Thermo Fisher). Cells were incubated in ice for 30 minutes and vortexed prior to flow cytometry. For measuring GFP expression in transiently transfected HEK cells, the 405 nm and 488 nm laser lines were used on the ZE5 flow cytometer (Biorad Inc, CA). Single live cells were used for analysis and gating schemes are furnished in the SI. At least 10,000 events were collected for each treatment condition.

### Toxicity Studies

Transfection was performed in 48-well plates according to the usual procedures. Two days after transfection, cell culture media was replaced with a 2% solution of CCK-8 (Dojindo) in Fluorbrite. Thereafter, cells were incubated for 4 hours at 37 C and 5% CO2 and absorbance measure at 450 nm at a gain of 90 using the Synergy H1 plate reader (Biotek, CA). Measurements of the CCK-8 solution without cells were collected and this blank reading was subtracted from all data points. Absorbance values were normalized to untreated cells. Three wells were employed for each treatment condition.

### HDR Experiments

For homology-directed repair, HEK293 cells were co-transfected with a mixture of RNP and donor plasmid payloads. The total mass of nucleic acids, comprising sgRNA and the DNA repair template, was fixed at either 1.5 μg per well or 2 μg for a 24-well plate. However their weight ratio was varied systematically from 2:1 to 1:5 in order to identify the formulation conditions that would maximize the frequency of HDR events. 2 μg loading per well and 1:2 w/w ratio of sgRNA:pDNA were identified as the optimal conditions using flow cytometric measurements.

In a typical experiment, RNP complexes were annealed by adding sgRNA solution to spCas9 solution in equal volumes. To assemble RNPs, spCas9 (Aldevron, ND,) and sgRNA (synthego, CA) solutions were prepared at concentrations of 0.039 mg/mL and 0.19 mg/mL respectively and the ribonucleoprotein formed by slow addition of sgRNA to spCas9 and annealing for 15 minutes. Within 15 minutes of RNP formation, an equal volume of the donor plasmid solution was added and allowed to equilibrate for 5 minutes. The polymer solution (diluted to the desired N/P ratio in D-PBS) was slowly introduced into an equal volume of the payload mixture and incubated for 45 minutes at ambient temperature. Finally, this mixture was diluted in twice the volume of OptiMEM and added slowly to cells. Cells were plated 24 hours prior to transfection at a density of 50,000 cells/mL. DMEM supplemented with HI-FBS would be added 4 hours after transfection and cell culture replaced 24 hours after transfection. Cells were regularly passaged while approaching 80% confluency (roughly every 2 days) before being analyzed using flow cytometry on the seventh day after transfection.

To efficiently explore a vast multidimensional experimental space proffered by cationic polymers, parallelized experimentation and statistical modeling were combined to accelerate the discovery of synthetic vehicles for genome editing payloads. A chemically diverse copolymer library consisting of 43 combinatorially designed polymeric variants was synthesized, thoroughly characterized, and rapidly screened for ribonucleoprotein delivery. High-throughput screening, aided by image cytometry, identified a hit polymer DIP50H50 within this chemical space, a structure that was found to outperform all four commercial RNP delivery reagents it was benchmarked against. Fluorescence read-outs were validated with Sanger sequencing to quantify indel formation during NHEJ editing. With an editing efficiency of 58%, which is twice as high as those observed in JetCRISPR and Lipofectamine CRISPRMAX, DIP50H50 is an exciting copolymeric prospect for implementing ex vivo editing through chemically defined vectors.

Further, DIP50H50 proved to be a versatile delivery vehicle capable of co-delivering plasmid DNA and RNP payloads to mediate precise gene editing via homology directed repair pathways, and to deliver plasmids to promote efficient transient transfection. By deploying high-throughput experimental workflows such as high content image analysis and parallel polymer synthesis, an extensive suite of physicochemical characterization data such as composition, molecular weight, pKa, polyplex diameter, RNP binding, electrokinetic data, as well as key biological readouts such as toxicity and uptake could be readily acquired, allowing us to systematically examine correlations between chemical structure, polymer properties and biological performance. High-throughput evaluation of editing efficiency, cellular viability and internalization formed the basis for statistical learning models that captured key biophysical trends: while cellular toxicity and RNP internalization were largely independent of polymer hydrophobicity and the extent cooperative deprotonation, these two polymer attributes were critical determinants of whether efficient RNP delivery would be achieved.

Since DIP50H50 emerged as a potent vector for RNP delivery in Examples 1-2, these results can be interpreted to indicate that in some examples the polymer design spaces for pDNA and RNP payloads overlap significantly. To probe this possibility, data science tools were applied to unravel the relationship between polymer attributes, type of payload, and biological outcomes such transfection efficiency, toxicity and uptake observed. Although the analysis established that the design rules for RNP and pDNA delivery were substantially congruent, hydrophobic interactions appeared to be far more consequential for efficient RNP delivery than for pDNA transfection. Unlike with RNP delivery where electrostatic considerations were negligible, pDNA polyplexes relied on both electrostatic and hydrophobic interactions to facilitate highly efficient pDNA transfection. Combinatorially designed libraries across multiple payloads can be used not only to uncover novel polymeric vectors for multimodal delivery applications, but also provides a robust framework for elucidation of payload-specific structure-function relationships.

Various embodiments of the invention have been described. These and other embodiments are within the scope of the following claims.

1: A compound, comprising:
a copolymer comprising a first (meth)acryl monomeric unit with a cationic functional group R₁ and a second (meth)acryl monomeric unit with a neutral hydrophilic functional group R₂;
wherein the cationic functional group R₁ is chosen from amino groups and alkylamino groups, and the neutral functional group R₂ is chosen from polyethylene glycol (PEG), hydroxyl (OH), phosphorylcholine (PC), and mixtures and combinations thereof; and
a biological agent associated with the copolymer.

2: The compound of claim 1, wherein R₁ is an alkylamino group.

3: The compound of claim 2, wherein the alkylamino group has a pKa of about 8 to about 10.

4: The compound of claim 2, wherein the alkylamino group is chosen from diethylamino, dimethylamino, diisopropylamino, and mixtures and combinations thereof.

5: The compound of claim 1, wherein R₂ comprises a hydroxyl group.

6: The compound of claim 1, wherein the first cationic monomeric unit is chosen from amino ethyl methacrylate (AEMA), 2-(diethylamino) ethyl methacrylate (DEAEMA), 2-dimethylamino ethyl methacrylate (DMAEMA), and 2-(diisopropylamino)ethyl methacrylate (DIPAEMA), and mixtures and combinations thereof.

7: The compound of claim 1, wherein the second neutral monomeric unit is chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof.

8: The compound of claim 1, wherein the copolymer comprises p(DIPAEMAₘ-st-HEMAₙ).

9: The compound of claim 8, wherein the copolymer is chosen from p(DIPAEMA₆₁-st-HEMA₃₃), p(DIPAEMA₅₂-st-HEMA₅₀), p(DIPAEMA₂₇-st-HEMA₅₀), and mixtures and combinations thereof.

10: The compound of claim 9, wherein the copolymer is p(DIPAEMA₅₂-st-HEMA₅₀).

11: The compound of claim 8, wherein the copolymer has a M_{w} of about 15 kDa to about 25 kDa.

12: The copolymer of claim 8, wherein the copolymer has a pKa of about 6.4 to about 7.3.

13: The copolymer of claim 8, wherein the copolymer has a ζ-potential of about −1 mV to about 20 mV.

14: The compound of claim 1, wherein the biological agent is entangled within the copolymer.

15: The compound of claim 14, wherein the biological agent is bound via electrostatic attraction to the copolymer.

16: The compound of claim 1, wherein the biological agent is chosen from pDNA, RNP, and mixtures and combinations thereof.

17: A composition comprising a pharmaceutically acceptable aqueous liquid carrier and the compound of claim 1.

18-41. (canceled)

42: A non-viral polyplex comprising a copolymer and a biological agent associated with the copolymer, wherein the copolymer comprises:
a cationic monomeric unit chosen from amino ethyl methacrylate (AEMA), 2-(diethylamino) ethyl methacrylate (DEAEMA), 2-dimethylamino ethyl methacrylate (DMAEMA), and 2-(diisopropylamino)ethyl methacrylate (DIPAEMA), and mixtures and combinations thereof; and
a neutral monomeric unit chosen from 2-methacryloyloxyethylphosphorylcholine (MPC), polyethylene glycol methylethyl methacrylate (PEG-MEMA), hydroxyethylmethyl methacrylate (HEMA), and mixtures and combinations thereof; and
wherein the biological agent associated with the copolymer is chosen from pDNA, RNP, and mixtures and combinations thereof.

43: The non-viral polyplex of claim 42, wherein the copolymer comprises p(DIPAEMAₘ-st-HEMAₙ).

44: The non-viral polyplex of claim 43, wherein the copolymer is chosen from p(DIPAEMA₆₁-st-HEMA₃₃), p(DIPAEMA₅₂-st-HEMA₅₀), p(DIPAEMA₂₇-st-HEMA₅₀), and mixtures and combinations thereof.

45: The non-viral polyplex of claim 44, wherein the copolymer is p(DIPAEMA₅₂-st-HEMA₅₀).

46: The non-viral polyplex of claim 42, wherein the polyplex is in an aqueous solution.

47: The non-viral polyplex of claim 46, wherein the aqueous solution is a pharmaceutically acceptable liquid carrier.

48-57. (canceled)
